(19) United States
(12) Patent Application Publication
(10) Pub. No.: US 2021/0356462 A1
(43) Pub. Date: Nov. 18, 2021

(54) MULTIPLEX ASSAY

(71) Applicant: Essenlix Corporation, Monmouth Junction, NJ (US)

(72) Inventors: Stephen CHOU, Princeton, NJ (US); Wei DING, Princeton, NJ (US); Ji LI, Princeton, NJ (US)

(73) Assignee: Essenlix Corporation, Monmouth Junction, NJ (US)

(21) Appl. No.: 17/295,818

(22) PCT Filed: Nov. 20, 2019
(86) PCT No.: PCT/US2019/062511

§ 371 Date: May 20, 2021

Related U.S. Application Data
(60) Provisional application No. 62/769,947, filed on Nov. 20, 2018.

(51) Int. Cl. G01N 33/543; B01L 3/00

(52) U.S. Cl. CPC G01N 33/54366; B01L 2200/16; B01L 3/5088

(57) ABSTRACT

The present disclosure provides devices, methods, and systems for performing cellular, biological, or chemical assays where a sample can have a plurality of analytes.

### CROSS-REFERENCE TO RELATED APPLICATIONS

This application is a National Stage entry (§ 371) application of International Application No. PCT/US2019/062511, filed on Nov. 20, 2019, which claims the benefit of priority of U.S. Provisional Patent Application No. 62/769,947, filed on Nov. 20, 2018, which is relied upon and incorporated herein by reference in its entirety. The entire disclosure of any publication or patent document mentioned herein is entirely incorporated by reference.

### FIELD

The present disclosure is related to the field of bio/chemical sampling, sensing, assays and applications. Particularly, the present invention is related to bio/chemical assays, including how to separate a certain component from a composite liquid sample and obtain the liquid sample without the component therein and/or extract the component from the sample.

### BACKGROUND

Multiplexed biomarker measurement can provide knowledge of the condition of a subject. For example, when monitoring the effects of a drug, two or more biomarkers can be measured in parallel. In a Point-of-Care (POC) device, the number of assays that can be performed in parallel is often limited by the size of the device and the volume of the sample to be analyzed. A POC device capable of performing multiplexed assays on a small sample is desirable.

### SUMMARY

In certain embodiments of the present disclosure, a device for analyzing a sample can comprise: a first plate, comprising a first storage site having a first reagent configured to bind to a first analyte, and a second storage site having a second reagent configured to bind to a second analyte; and a second plate, movable relative to the first plate into different configurations including an open configuration and a closed configuration. In certain embodiments, in the open configuration, the first plate and the second plate are at least partially separated, and at least one of the first plate and the second plate receives deposition of a sample containing or suspected of containing the first analyte and the second analyte. In certain embodiments, in the closed configuration, at least a portion of the deposited sample is compressed into a layer of uniform thickness in contact with the first plate and the second plate to form a sample thickness. In certain embodiments, the first storage site and the second storage site are fluidically connected by the deposited sample and are separated from each other by a separation distance, and the separation distance is greater than (i) a distance that the first reagent contained in the first storage site can diffuse to the second storage site within a period of time, or (ii) a distance that the second reagent contained in the second storage site can diffuse to the first storage site within a period of time. In certain embodiments, the first storage site and the second storage site are fluidically connected by the deposited sample and are separated from each other by a separation distance, wherein the separation distance is governed by the formula:

Separation Distance≥10×Sample Thickness.

In certain embodiments of the present disclosure, a device for analyzing a sample comprises: a first plate, comprising a first storage site having a first reagent configured to bind to a first analyte. In certain embodiments of the present disclosure, a device for analyzing a sample comprises a second storage site having a second reagent configured to bind to a second analyte. In certain embodiments of the present disclosure, a device for analyzing a sample comprises a second plate movable relative to the first plate into different configurations including an open configuration and a closed configuration. In certain embodiments of the present disclosure, in the open configuration, the first plate and the second plate are at least partially separated, and at least one of the first plate and the second plate receives deposition of a sample containing or suspected of containing the first analyte and the second analyte. In certain embodiments of the present disclosure, in the closed configuration, at least a portion of the deposited sample is compressed into a layer of uniform thickness in contact with the first plate and the second plate to form a sample thickness. In certain embodiments of the present disclosure, the first storage site and the second storage site are fluidically connected by the deposited sample and are separated from each other by a separation distance, and the separation distance is greater than a distance that the first reagent contained in the first storage site can diffuse to the second storage site within a period of time, or a distance that the second reagent contained in the second storage site can diffuse to the first storage site within a period of time.

In certain embodiments of the present disclosure, a device for analyzing a sample, comprises a first plate comprising a first storage site having a first reagent configured to bind to a first analyte. In certain embodiments of the present disclosure, a device for analyzing a sample comprises a first plate comprising a second storage site having a second reagent configured to bind to a second analyte. In certain embodiments of the present disclosure, a device for analyzing a sample comprises a second plate movable relative to said first plate into different configurations including an open configuration and a closed configuration. In certain embodiments of the present disclosure, in the open configuration, the first plate and the second plate are at least partially separated, and at least one of the first plate and the second plate receives deposition of a sample containing or suspected of containing the first analyte and the second analyte. In certain embodiments of the present disclosure, in the closed configuration, at least a portion of the deposited sample is compressed into a layer of uniform thickness in contact with the first plate and the second plate to form a sample thickness. In certain embodiments of the present disclosure, the first storage site and the second storage site are fluidically connected by the deposited sample and are separated from each other by a separation distance, and wherein the separation distance is governed by the formula of: Separation Distance≥Sample Thickness.

In certain embodiments of the present disclosure, a device for analyzing a sample comprises a first plate comprising a first storage site having a first reagent configured to bind to a first analyte. In certain embodiments of the present disclosure, a device for analyzing a sample comprises a second plate comprising a second storage site having a second reagent configured to bind to a second analyte, wherein the second plate is movable relative to the first plate into different configurations including an open configuration and a closed configuration. In certain embodiments of the present disclosure, in the open configuration, the first plate and the second plate are at least partially separated, and at least one of the first plate and the second plate receives deposition of a sample containing or suspected of containing the first analyte and the second analyte. In certain embodiments of the present disclosure, in the closed configuration, at least a portion of the deposited sample is compressed into a layer of uniform thickness in contact with the first plate and the second plate to form a sample thickness. In certain embodiments of the present disclosure, the first storage site and the second storage site are fluidically connected by the deposited sample and are separated from each other by a separation distance, and the separation distance is greater than a distance that the first reagent contained in the first storage site can diffuse to the second storage site within a period of time, or a distance that the second reagent contained in the second storage site can diffuse to the first storage site within a period of time.

In certain embodiments of the present disclosure, a device for analyzing a sample comprises a first plate comprising a first storage site having a first reagent configured to bind to a first analyte. In certain embodiments of the present disclosure, a device for analyzing a sample comprises a second plate comprising a second storage site having a second reagent configured to bind to a second analyte, wherein the second plate is movable relative to the first plate into different configurations including an open configuration and a closed configuration. In certain embodiments of the present disclosure, in the open configuration, the first plate and the second plate are at least partially separated, and at least one of the first plate and the second plate receives deposition of a sample containing or suspected of containing the first analyte and the second analyte. In certain embodiments of the present disclosure, in the closed configuration, at least a portion of the deposited sample is compressed into a layer of uniform thickness in contact with the first plate and the second plate to form a sample thickness. In certain embodiments of the present disclosure, the first storage site and the second storage site are fluidically connected by the deposited sample and are separated from each other by a separation distance, and wherein the separation distance is governed by the formula of: Separation Distance≥Sample Thickness.

In certain embodiments of the present disclosure, at least one of the plates is transparent. In certain embodiments of the present disclosure, the first plate and the second plate are made of a material selected from, for example, polystyrene, PMMA, PC, COC, COP, or a combination thereof. In certain embodiments of the present disclosure, at least one of the first plate and the second plate have a thickness in the range of 20 μm to 250 μm. In certain embodiments of the present disclosure, at least one of the first plate and the second plate have a Young's modulus in the range 0.1 to 5 GPa. In certain embodiments of the present disclosure, the thickness of the first plate or the second plate times the Young's modulus of the first plate or the second plate is from 60 to 750 GPa-um. In certain embodiments of the present disclosure, the layer of uniform thickness is uniform over a lateral area that is at least 1 mm². In certain embodiments of the present disclosure, the layer of uniform thickness sample has a thickness uniformity of up to +/−5%. In certain embodiments of the present disclosure, the uniform thickness between the first plate and the second plater, in the closed configuration, is less than 200 μm. In certain embodiments of the present disclosure, the uniform thickness between the first plate and the second plate, in the closed configuration, is between 1 μm and 30 μm. In certain embodiments of the present disclosure, at least one of the plates has a plurality of spacers affixed thereon. In certain embodiments of the present disclosure, the spacers are made of a material selected from , for example, polystyrene, PMMA, PC, COC, COP, or a combination thereof. In certain embodiments of the present disclosure, the spacers have a pillar shape. In certain embodiments of the present disclosure, the spacers have a pillar shape, and the sidewall corners of the spacers have a round shape with a radius of curvature at least 1 μm. In certain embodiments of the present disclosure, the spacers are pillars with a cross-sectional shape selected from, for example, round, polygonal, circular, triangular, square, rectangular, oval, elliptical, or a super-possitional combination thereof. In certain embodiments of the present disclosure, the spacers have a substantially flat top surface. In certain embodiments of the present disclosure, the spacers have a substantially uniform cross-section. In certain embodiments of the present disclosure, the spacers have a predetermined substantially uniform height. In certain embodiments of the present disclosure, the spacers have a constant inter-spacer distance. In certain embodiments of the present disclosure, the constant inter-spacer distance is from about 1 μm to 120 μm. In certain embodiments of the present disclosure, the spacers have a periodic inter-spacer distance. In certain embodiments of the present disclosure, for each spacer, the ratio of the lateral dimension of the spacer to its height is at least 1. In certain embodiments of the present disclosure, the spacers have a filling factor of 1% or higher, wherein the filling factor is the ratio of the spacer lateral contact area to the total lateral plate area of one plate. In certain embodiments of the present disclosure, the Young's modulus of the spacers times filling factor of the spacers is equal or larger than 20 MPa, wherein the filling factor is the ratio of the spacer lateral contact area to the lateral total plate area of one plate. In certain embodiments of the present disclosure, the spacers have a density of at least 1,000/mm². In certain embodiments of the present disclosure, the first analyte and the second analyte are the same. In certain embodiments the first reagent comprises a cell lysis agent. In certain embodiments of the present disclosure, the cell lysis agent is selected from an ethoxylated nonylphenol non-ionic surfactant, sodium deoxycholate, sodium dodecyl sulfate, ammonium-chloride-potassium (ACK) buffer, a zwittenonic detergent of the formula C₁₉H₄₁NO₃S, a zwitterionic detergent, or a saponin. In certain embodiments of the present disclosure, the first reagent is configured to perform a first assay, and the second reagent is configured to perform a second assay. In certain embodiments of the present disclosure, the first assay and the second assay are independently selected from the group consisting of an optical assay, a colorimetric assay, a mercurimetric assay, a luminescent assay, an electro-luminescent assay, a chemical-luminescent assay, a nonlinear optical assay, an electrical assay, a capacitive measurement assay, a resistive measurement assay, an impedance measurement assay, a chemical assay, and a mechanical assay. In certain embodiments of the present disclosure, the device further comprises a third storage site comprising a third reagent for binding to a third target analyte. In certain embodiments of the present disclosure, the third storage site is separated from the first storage site and the second storage site by the separation distance. In certain embodiments of the present disclosure, the third storage site is fluidically connected to the first storage site and the second storage site by the deposited sample. In certain embodiments of the present disclosure, the period of time is 1 second, 5 seconds, 10 seconds, 20 seconds, 30 seconds, 40 seconds, 50 seconds, 1 minute, 2 minutes, 3 minutes, 4 minutes, 5 minutes, 10 minutes, 15 minutes, 30 minutes, or greater than 30 minutes, including intermediate values and ranges. In certain embodiments of the present disclosure, the separation distance is 5 micrometers (um), 10 um, 25 um, 50 um, 100 um, 150 um, 200 um, 250 um, 300 um, 400 um, 500 um, 750 um, 1 millimeter, 2 mm, 3 mm, 4 mm, or 5 mm, including intermediate values and ranges. In certain embodiments of the present disclosure, the second storage site surrounds the first storage site. In certain embodiments of the present disclosure, the first storage site and the second storage site have a shape that are independently selected from round, polygonal, circular, triangular, square, rectangular, pentagonal, hexagonal, oval, elliptical, or a combination thereof. In certain embodiments of the present disclosure, the separation distance is governed by the formula of: Separation Distance≥2×Sample Thickness. In certain embodiments of the present disclosure, the separation distance is governed by the formula of: Separation Distance≥5×Sample Thickness. In certain embodiments of the present disclosure, the separation distance is governed by the formula of: Separation Distance≥10×Sample Thickness.

In certain embodiments of the present disclosure, a method for analyzing a liquid sample comprises obtaining a sample that contains or is suspected to contain a first analyte and a second analyte. In certain embodiments of the present disclosure, a method for analyzing a liquid sample comprises obtaining the device of any embodiment of the present disclosure. In certain embodiments of the present disclosure, a method for analyzing a liquid sample comprises depositing the sample on one or both of the plates when the plates are configured in an open configuration, wherein the open configuration is a configuration in which the two plates are either partially or completely separated apart and the spacing between the plates is not regulated by the spacers. In certain embodiments of the present disclosure, a method for analyzing a liquid sample comprises bringing the two plates together into a closed configuration; in which at least part of the sample forms a layer of substantially uniform thickness that is confined by the sample contact surfaces of the plates. In certain embodiments of the present disclosure, the substantially uniform thickness of the layer is regulated by the spacers and the plates in the closed configuration. In certain embodiments of the present disclosure, a method for analyzing a liquid sample comprises analyzing at least one of the first analyte and the second analyte in the layer of uniform thickness while the plates are in the closed configuration.

In certain embodiments of the present disclosure, a method for analyzing a liquid sample comprises obtaining a sample that contains or is suspected to contain a first analyte and a second analyte. In certain embodiments of the present disclosure, a method for analyzing a liquid sample comprises obtaining a device comprising a first plate; a second plate; and a first storage site comprising a first reagent and a second storage site comprising a second reagent, wherein each of the first storage site and the second storage site are (A) disposed on one of the first plate and the second plate, and (B) fluidically connected by a sample. In certain embodiments of the present disclosure, a method for analyzing a liquid sample comprises depositing the sample on one or both of the plates when the plates are configured in an open configuration, wherein the open configuration is a configuration in which the two plates are either partially or completely separated apart and the spacing between the plates is not regulated by the spacers. In certain embodiments of the present disclosure, a method for analyzing a liquid sample comprises bringing the two plates together into a closed configuration; in which at least part of the sample forms a layer of substantially uniform thickness that is confined by the sample contact surfaces of the plates, wherein the substantially uniform thickness of the layer is regulated by the spacers and the plates. In certain embodiments of the present disclosure, a method for analyzing a liquid sample comprises analyzing at least one of the first analyte and the second analyte in the layer of uniform thickness while the plates are in the closed configuration. In certain embodiments of the present disclosure, the analyzing comprises making or taking one or more images with an imager such as a camera, a CCD, or like devices, and optional optics, and which imager is part of an apparatus for analyzing the sample for analytes between the plates in the device. In certain embodiments of the present disclosure, the analyzing comprises (i) analyzing one or more first images from a first field of view within the first storage site, and (ii) analyzing one or more second images from a second field of view within the second storage site, wherein the first field of view and the second field of view are separated by a separation distance, and wherein the separation distance is greater than a distance that the first reagent can diffuse to the second field of view within a period of time, or a distance that the second reagent can diffuse to the first field of view within a period of time.

In certain embodiments of the present disclosure, the method further comprises determining a relevant volume, wherein the relevant volume is a product of a predetermined area and a thickness of the layer of uniform thickness at the closed configuration. In certain embodiments of the present disclosure, the method further comprises determining a concentration of first analyte and/or the second analyte in a relevant volume of sample.

### BRIEF DESCRIPTION OF THE DRAWINGS

A skilled artisan will understand that the drawings, described below, are for illustration purposes only. In some Figures, the drawings are in scale. In the figures that present experimental data points, the lines that connect the data points are for guiding a viewing of the data only and have no other means. For clarity purposes, some elements are enlarged when illustrated in the Figures. It should be noted that the Figures do not intend to show the elements in strict proportion. The dimensions of the elements should be delineated from the descriptions provided herein and incorporated by reference. The drawings are not intended to limit the scope of the present invention in any way.

FIG. 1 illustrates a prospective view of an exemplary embodiment of the present disclosure in which a first plate has two storage sites separated by a separation distance (D).

FIG. 2 illustrates a side view of an exemplary embodiment of the present disclosure in which a first plate has two storage sites separated by a separation distance (D), wherein the separation distance is determined as a function of the sample thickness (T) and a predetermined separation factor. The separation factor is equal to the separation distance (D) divided by the sample thickness. The separation factor is greater than or equal to a predetermined value.

FIG. 3A illustrates a side view of an exemplary embodiment of the present disclosure in which the separation factor is greater than or equal to a predetermined value. In particular, a first plate has two storage sites separated by a separation distance (D), wherein the separation distance is large enough such that analytes that have contacted the first reagent do not mix with analytes that have contacted the second reagent.

FIG. 3B illustrates a side view of an exemplary embodiment of the present disclosure in which the separation factor is greater than or equal to a predetermined value. In particular, a first plate has two storage sites separated by a separation distance (D), wherein the separation distance is large enough such that first analytes that have contacted the first reagent do not contact the second storage site, and second analytes that have contacted the second reagent do not contact the first storage site.

FIG. 4 illustrates a side view of an exemplary embodiment of the present disclosure in which the separation factor is less than a predetermined value. In particular, a first plate has two storage sites separated by a separation distance (D), wherein the separation distance is not large enough, thereby resulting in substantial mixing such that first analytes that have contacted the first reagent contact the second storage site, and/or second analytes that have contacted the second reagent contact the first storage site.

FIG. 5A illustrates a side view of an exemplary embodiment of the present disclosure in which the separation factor is greater than or equal to a predetermined value. In particular, a first plate has a first storage site, a second plate has a second storage site, and the two storage sites separated by a separation distance (D), wherein the separation distance is large enough such that analytes that have contacted the first reagent do not mix with analytes that have contacted the second reagent.

FIG. 5B illustrates a side view of an exemplary embodiment of the present disclosure in which the separation factor is greater than or equal to a predetermined value. In particular, a first plate has a first storage site, a second plate has a second storage site, and the two storage sites separated by a separation distance (D), wherein the separation distance is large enough such that first analytes that have contacted the first reagent do not contact the second storage site, and second analytes that have contacted the second reagent do not contact the first storage site.

FIG. 6 illustrates a side view of an exemplary embodiment of the present disclosure in which the separation factor is less than a predetermined value. In particular, a first plate has a first storage site, a second plate has a second storage site, and the two storage sites separated by a separation distance (D), wherein the separation distance is not large enough, thereby resulting in substantial mixing such that first analytes that have contacted the first reagent contact the second storage site, and/or second analytes that have contacted the second reagent contact the first storage site.

FIG. 7 illustrates a side view of an exemplary embodiment of the present disclosure in which a first plate has two storage sites that overlap. Each storage site comprises a field of view within which a sample is analyzed, and the fields of view are separated by a separation distance that is large enough such that analytes that have contacted a first reagent from the first storage site do not mix with analytes that have contacted a second reagent from the second storage site.

FIG. 8 illustrates a prospective view of an exemplary embodiment of the present disclosure in which a first plate has four storage sites separated by a separation distance (D).

### DETAILED DESCRIPTION

The following detailed description illustrates certain embodiments of the invention by way of example and not by way of limitation. If any, the section headings and any subtitles used herein are for organizational purposes only and are not to be construed as limiting the subject matter described in any way. The contents under a section heading and/or subtitle are not limited to the section heading and/or subtitle, but apply to the entire description of the present invention.

Definitions

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The term “diffusion parameter” or “DP” as used herein refers to a parameter that is equal to \sqrt{Dt}, wherein D is the diffusion constant of the analyte in the sample and the t is the intended assay time (i.e., the diffusion parameter is equal to the square-root of the diffusion constant of the analyte in the sample and multiplied by the intended assay time); wherein the intended assay time is a time parameter. For example, if the diffusion constant of the analyte in the sample is 1×10⁻⁷ cm²/s, and the intended assay time (t) is 60 sec, then the diffusion parameter is 24 μm (microns). Some of the common analyte diffusion constants are: IgG in PBS: 3×10⁻⁷ cm²/s, IgG in blood: 1×10⁻⁷ cm²/s, and 20 bp DNA in blood: 4×10⁻⁷ cm²/s.

The term “sample” as used herein relates to a material or mixture of materials containing one or more analytes or entities of interest. In particular embodiments, the sample can be obtained from a biological sample such as cells, tissues, bodily fluids, or stool. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine, and exhaled condensate. In particular embodiments, a sample can be obtained from a subject, e.g., a human, and it can be processed prior to use in the subject assay. For example, prior to analysis, the protein/nucleic acid can be extracted from a tissue sample prior to use, for which methods are known. In particular embodiments, the sample can be a clinical sample, e.g., a sample collected from a patient.

The term “analyte” refers to a molecule (e.g., a protein, peptides, DNA, RNA, nucleic acid, or other molecule), cells, tissues, viruses, or nanoparticles with different shapes.

The term “assaying” refers to testing a sample to detect the presence and/or abundance of an analyte.

As used herein, the terms “determining,” “measuring,” “assessing,” and “assaying” can be used interchangeably and include both quantitative and qualitative determinations.

A subject can be any human or non-human animal. A subject can be a person performing the instant method, a patient, a customer in a testing center, and like persons.

An “analyte,” as used herein is any substance that is suitable for testing in the present invention.

As used herein, a “diagnostic sample” refers to any biological sample that is a bodily byproduct, such as bodily fluids, that has been derived from a subject. The diagnostic sample can be obtained directly from the subject in the form of liquid, or can be derived from the subject by first placing the bodily byproduct in a solution, such as a buffer. Exemplary diagnostic samples include, but are not limited to, saliva, serum, blood, sputum, urine, sweat, lacrima, semen, feces, breath, biopsies, mucus, and like samples.

As used herein, an “environmental sample” refers to any sample that is obtained from the environment. An environmental sample can include liquid samples from a river, lake, pond, ocean, glaciers, icebergs, rain, snow, sewage, reservoirs, tap water, drinking water, and like sources; solid samples from soil, compost, sand, rocks, concrete, wood, brick, sewage, and like sources; and gaseous samples from the air, underwater heat vents, industrial exhaust, vehicular exhaust, and like sources. Typically, samples that are not in liquid form can be converted to liquid form before analyzing the sample with the present invention.

As used herein, a “foodstuff sample” refers to any sample that is suitable for animal consumption, e.g., human consumption. A foodstuff sample can include raw ingredients, cooked food, plant and animal sources of food, preprocessed food and partially or fully processed food, and like samples. Typically, samples that are not in liquid form are converted to liquid form before analyzing the sample with the present invention.

The term “diagnostic,” as used herein, refers to the use of a method or an analyte for identifying, predicting the outcome of, and/or predicting treatment response of a disease or condition of interest. A diagnosis can include predicting the likelihood of or a predisposition to having a disease or condition, estimating the severity of a disease or condition, determining the risk of progression in a disease or condition, assessing the clinical response to a treatment, and/or predicting the response to treatment.

A “biomarker,” as used herein, is any molecule or compound that is found in a sample of interest and that is known to be diagnostic of or associated with the presence of or a predisposition to a disease or condition of interest in the subject from which the sample is derived. Biomarkers include, but are not limited to, polypeptides or a complex thereof (e.g., antigen, antibody), nucleic acids (e.g., DNA, miRNA, mRNA), drug metabolites, lipids, carbohydrates, hormones, vitamins, and like entities, that are known to be associated with a disease or condition of interest.

A “condition” as used herein with respect to diagnosing a health condition, refers to a physiological state of mind or body that is distinguishable from other physiological states. A health condition cannot be diagnosed as a disease in some cases. Exemplary health conditions of interest include, but are not limited to, nutritional health; aging; exposure to environmental toxins, pesticides, herbicides, synthetic hormone analogs; pregnancy; menopause; andropause; sleep; stress; prediabetes; exercise; fatigue; chemical balance; and like conditions. The term “biotin moiety” refers to an affinity agent that includes biotin or a biotin analogue such as desthiobiotin, oxybiotin, 2′-iminobiotin, diaminobiotin, biotin sulfoxide, biocytin, and like moieties. Biotin moieties bind to streptavidin with an affinity of at least 10⁻⁸ M. A biotin affinity agent can also include a linker, e.g., —LC-biotin, —LC-LC-Biotin, —SLC-Biotin, or —PEGn-Biotin, where n is from 3 to 12.

The term “marker”, as used in describing a biological sample, refers to an analyte whose presence or abundance in a biological sample is correlated with a disease or condition.

The term “amplify” refers to an increase in the magnitude of a signal, e.g., at least a 10-fold increase, at least a 100-fold increase at least a 1,000-fold increase, at least a 10,000-fold increase, or at least a 100,000-fold increase in a signal.

The term “entity” refers to, but not limited to proteins, peptides, DNA, RNA, nucleic acid, molecules (small or large), cells, tissues, viruses, nanoparticles with different shapes, that would bind to a “binding site”. The entity includes, for example, a capture agent, a detection agent, and a blocking agent. The “entity” includes the “analyte”, and the two terms can be used interchangeably.

The term “target analytes” or “target entity” refers to a particular analyte that will be specifically analyzed (i.e., detected, quantified, or both), or a particular entity that will be specifically bound to the binding site.

The term “smart phone”, “mobile phone”, or “mobile communication device”, can be used interchangeably, and refer to the type of phone device that has a camera and communication hardware and software that can take an image using the camera, manipulate the image taken by the camera, and communicate data to a remote place. In some embodiments, the smart phone has a flashlight or a light source for illuminating the sample.

The term “light” refers to, unless specifically specified, an electromagnetic radiation with various wavelength.

The term “storage site” refers to a site of an area on a plate, wherein the site contains reagents to be added into a sample, and the reagents are capable of dissolving into the sample that is in contract with the reagents and diffusing into the sample.

The term “relevant” means that something is relevant to detection of analytes, quantification and/or control of analyte or entity in a sample or on a plate, or quantification or control of reagent to be added to a sample or a plate.

The term “variation” of a quantity refers to the difference between the actual value and the desired value or the average of the quantity. And the term “relative variation” of a quantity refers to the ratio of the variation to the desired value or the average of the quantity. For example, if the desired value of a quantity is Q and the actual value is (Q+Δ), then the Δ is the variation and the Δ/(Q+Δ) is the relative variation. The term “relative sample thickness variation” refers to the ratio of the sample thickness variation to the average sample thickness.

The term “compressed open flow (COF)” refers to a method that changes the shape of a flowable sample deposited on a plate by (i) placing another plate on top of at least a part of the sample and (ii) then compressing the sample between two plates by pushing the two plates towards each other; wherein the compression reduces a thickness of at least a part of the sample and makes the sample flow into open spaces between the plates.

The term “compressed regulated open flow” or “CROF” (or “self-calibrated compressed open flow” or “SCOF” or “SCCOF”) refers to a particular type of COF, wherein the final thickness of a part or an entire sample after the compression is “regulated” by spacers, wherein the spacers are placed or located between the two plates.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible. One skilled artisan will appreciate that the present invention is not limited in its application to the details of construction, the arrangements of components, category selections, weightings, pre-determined signal limits, or the steps set forth in the description or drawings herein. The invention is capable of other embodiments and of being practiced or being carried out in many different ways.

Multiplexed Biomarker Measurement

Many existing methodologies are not multiplexed. That is, optimization of analysis conditions and interpretation of results are performed in simplified single determination assays. However, this can be problematic. If symptoms are ambiguous, or indicative of any number of different disease organisms, a device or method capable of screening for numerous possible causative agents is highly desirable. Moreover, if symptoms are complex, possibly caused by multiple biomarkers (e.g., pathogens and/or analytes), an assay that functions as a “decision tree” which indicates with increasing specificity the biomarkers involved is valuable.

Multiplexing requires additional controls to maintain accuracy. False positive or negative results due to contamination, degradation of sample, presence of inhibitors or cross reactants, and inter/intra strand interactions should be considered when designing the analysis conditions. In particular, multiplexing using a single sample in a single sample chamber (e.g., wherein the multiplexed assays are not fluidically isolated from one another) can also be challenging. In certain embodiments of the present disclosure, multiplexing is performed by spacing storage sites (e.g., comprising one or more assay reagents) at a predetermined separation distance sufficient to prevent one or more reagents from a first storage site from interfering with one or more reagents from a second storage. In other words, a separation distance between the two storage sites is large enough to allow multiple assays to be performed within a single sample.

Embodiments of the present disclosure can include spacers that have a substantially uniform height, a nearly uniform cross-section (e.g., a pillar with straight sidewall), and planar (i.e., “flat”) tops, that are fixed to one or more of the plates in regular pattern in which the spacers are separated from one another by a consistent, defined, distance (i.e., not at random positions that are governed by Poisson statistics). During use, the spacers and plates are not significantly compressed or deformed in any dimension, at least while the plates are in the closed position and being pulled together by capillary force. In certain embodiments of the present disclosure, the spacer height and assay end point can be chosen to limit the amount of lateral diffusion of analytes during the assay. In these cases, such an assay (typically a binding assay) can be run in a very short time. In addition, the concentration of the analyte in the sample can be estimated very accurately, even though the entire sample cannot have been analyzed or can be of an unknown volume. In these embodiments, an assay can be stopped and/or assay results can be read at a time that is: i) equal to or longer to the time that it takes for a target entity to diffuse across the thickness of the uniform thickness layer at the closed configuration (i.e., shorter than the time that it would take for the analyte to vertically diffuse from one plate to the other); and ii) shorter than the time that it takes the target entity to laterally diffuse across the linear dimension of the predetermined area of the binding site (i.e., shorter than the time that it would take for the analyte to laterally diffuse from one side of the binding site to other). In such “local binding” configurations, the volume of the part of the sample from which data is obtained (the “relevant volume”) can be estimated reasonably accurately because it is the volume of the sample that is immediately above the analyzed area. Indeed, the volume of the part of the sample from which data is obtained can be known before the assay is initiated. Such “local binding” embodiments have an additional advantage in that the sample and, optionally, any detection reagents are pressed into a thin layer over a binding site and, as such, binding between any analytes and/or detection reagents should reach equilibrium more quickly than in embodiments in which the sample is not pressed into a thin layer, e.g., if a drop of sample is simply placed on top of a plate with the binding site. As such, in many cases, binding equilibrium can be reached in a matter of seconds rather than minutes and, as such, many assays, particularly binding assays, can be done very quickly, e.g., in less than a minute.

Furthermore, the “local binding” configuration allows one to perform multiplex assays without fluidically isolating the different reactions from one another. In other words, multiple assays can be done in an open environment, without the assays being walled off from one another (i.e., without fluidic isolation). For example, in local binding embodiments, two different analytes in the same sample can be assayed side-by-side and, because the assay is be stopped and/or the assay results are be read prior to diffusion of the one analyte from one assay area into the other, the absolute concentrations of those analytes in the sample can be determined separately from one another, even though they are not fluidically isolated from one another.

The ability to perform multiple assays on one sample, without fluidic isolation, by simply sandwiching a sample between two plates and performing the assay in a diffusion-limited way has several advantages. For example, the assays can be done by simply dropping a droplet of a sample (e.g., blood) of an unknown volume, spreading out the sample across the plates by pressing the plates together, incubating the sample for a period of time, and taking a reading from multiple sites in the device. In practicing this method, one does not need to transfer defined amounts of a sample into several chambers, which is difficult to implement without an accurate fluid transfer and/or measuring device. Moreover, the assay is extremely rapid for the reasons set out above. Further, because the plates do not need to be made with “walls” the manufacture of the device is straightforward. Finally, there is no requirement for ports in any of the plates, i.e., ports that could potentially be used for adding or removing sample or a reagent while the device is in closed position.

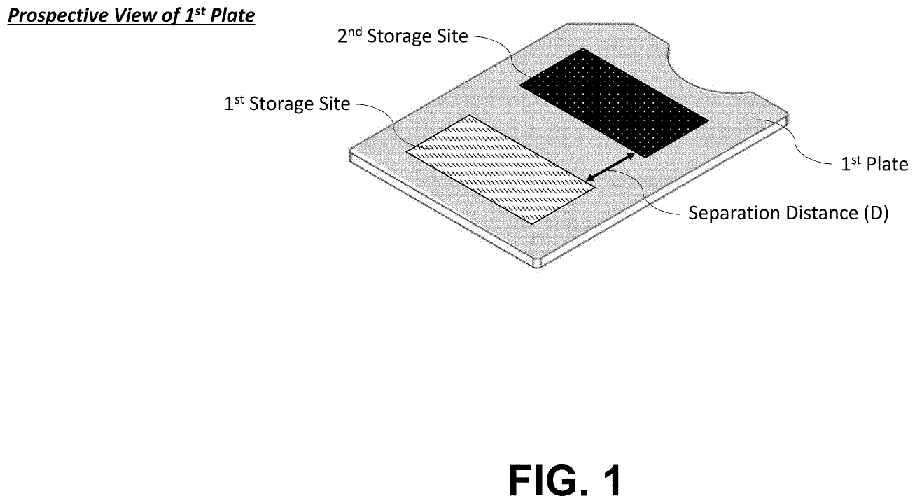

Separation Distance. The term “separation distance” can refer to a distance between two neighboring storage sites. In certain embodiments, the separation distance can refer to the shortest distance between two neighboring storage sites. In other embodiments, the separation distance can refer to an average distance between two neighboring storage sites. As illustrated in FIG. 1, a plate (e.g., a first plate) of a QMAX card can have a first storage site (having one or more first reagents and/or capture agents) and a second storage site (having one or more second reagents and/or capture agents) disposed thereon and separated by a separation distance (D).

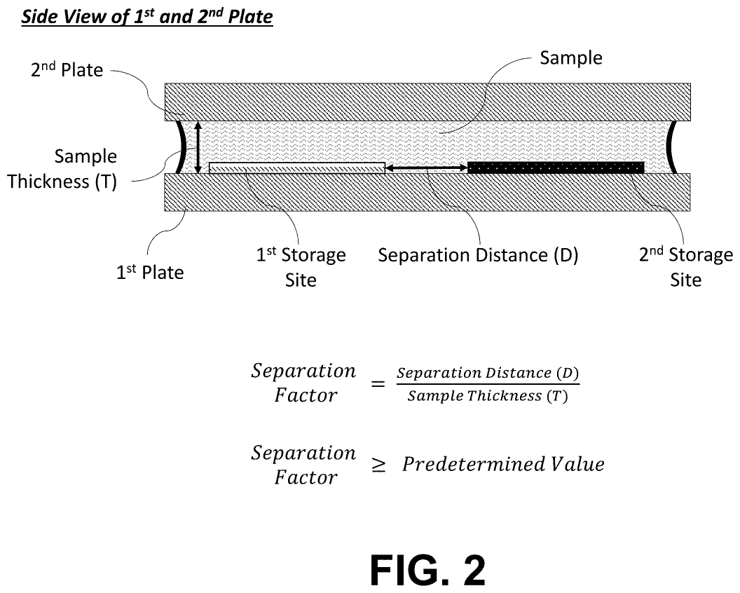

As illustrated in FIG. 2, the first storage site and the second storage site are not fluidically isolated (e.g., a single sample contacts both of the first storage site and the second storage site). To prevent interference and/or mixing between the reagents of first storage site and the second storage site, the first and second storage sites are separated by a separation distance. In certain embodiments, the separation distance is predetermined. In certain embodiments, the separation distance is predetermined based on the type of assay being performed (e.g., a colorimetric assay, a fluorescence-based assay, an electrical assay, and a mechanical assay). In certain embodiments, the separation distance can be determined as a product of: (i) a sample thickness (T) when the sample is compressed between the first plate and the second plate; and (ii) a separation factor. In certain embodiments, the separation factor can be, for example, 0.1, 0.5, 1, 2, 3, 4, 5, 6, 7, 8, 9, 10, 15, 20, 25, 50, or 100. For example, the separation distance can be determined as a product of 10 (e.g., the separation factor) and the sample thickness. In another example, the separation distance can be determined as a product of 5 (e.g., the separation factor) and the sample thickness. In certain embodiments, the separation distance (e.g., between neighboring storage sites) can be, for example, about 1 micron (um), about 5 um, about 10 um, about 25 um, about 50 um, about 100 um, about 250 um, about 500 um, about 750 um, about 1 millimeter (mm), about 2 mm, about 3 mm, about 4 mm, about 5 mm, about 6 mm, about 7 mm, about 8 mm, about 9 mm, about 10 mm, or about 20 mm. In certain embodiments, the separation distance (e.g., between neighboring storage sites) can be, for example, at least about 1 micron (um), at least about 5 um, at least about 10 um, at least about 25 um, at least about 50 um, at least about 100 um, at least about 250 um, at least about 500 um, at least about 750 um, at least about 1 millimeter (mm), at least about 2 mm, at least about 3 mm, at least about 4 mm, at least about 5 mm, at least about 6 mm, at least about 7 mm, at least about 8 mm, at least about 9 mm, at least about 10 mm, or at least about 20 mm. In certain embodiments, the separation distance can be between about 1 micron and about 20 mm. In certain embodiments, the separation distance can be between about 100 um and about 10 mm. In certain embodiments, the separation distance can be between about 500 um to and about 750 um.

In some embodiments, the separation distance is 1 um, 2 um, 3 um, 5 um, 10 um, 50 um, 100 um, 500 um, 1 mm, 2 mm, 3 mm, 5 mm, 10 mm or a range between any two of the values.

In some embodiments, the preferred separation distance is 50 um, 100 um, 200 um, 300 um, 500 um, 1 mm, 2 mm, or a range between any two of the values.

In some embodiments, the separation distance is greater than a product of the separation factor and “diffusion parameter” or “DP” refers to a parameter that is equal to \sqrt{Dt}, wherein D is the diffusion constant of the analyte in the sample and the t is the intended assay time (i.e. the diffusion parameter is equal to the square-root of the diffusion constant of the analyte in the sample multiplying the intended assay time); wherein the intended assay time is a time parameter.

In certain embodiments, the separation factor can be 0.1, 0.5, 1, 2, 3, 4, 5, 6, 7, 8, 9 ,10, 15, 20, 25, 50, 100 or a range between any two of the values.

In certain embodiments, the preferred separation factor is 1, 1.2, 1.5, 2, 3, 4, 5, or a range between any two of the values.

In certain embodiments, the intended assay time is 10 second, 30 second, 60 second, 90 second, 120 second, 180 second, 240 second, 300 second, 1000 second, 3600 second, 7200 second or a range between any two of the values.

In certain embodiments, the preferred intended assay time is 30 second, 60 second, 90 second, 120 second, 180 second, 240 second, 300 second or a range between any two of the values.

D is the diffusion constant of the analyte in the sample. Some examples of the common analyte diffusion constants are: IgG in PBS: 4×10⁻⁷ cm²/s, IgG in whole blood: 1×10⁻⁷ cm²/s, 20 bp sDNA in PBS: 15×10⁻⁷ cm²/s, 20 bp sDNA in blood: 4×10⁻⁷ cm²/s, 20 bp dDNA in blood: 11×10⁻⁷ cm²/s, 20 bp dDNA in blood: 2×10⁻⁷ cm²/s.

Some examples of the beads/particle/cells diffusion constants are: 20 nm diameter in PBS: 2×10⁻⁷ cm²/s, 20 nm diameter in whole blood: 0.5×10⁻⁷ cm²/s, 100 nm diameter in PBS: 0.4×10⁻⁷ cm²/s, 100 nm diameter in whole blood: 0.1×10⁻⁷ cm²/s, 1 um diameter in PBS: 0.04×10⁻⁷ cm²/s, 1 um diameter in whole blood: 0.01×10⁻⁷ cm²/s, 10 um diameter in PBS: 0.004×10⁻⁷ cm²/s, 10 um diameter in whole blood: 0.001×10⁻⁷ cm²/s.

In some embodiments, the intended assay time is 30 second, the analyte is IgG in PBS, the preferred separation distance is larger than 30 um, 34 um, 40 um, 50 um, 80 um, 100 um.

In some embodiments, the intended assay time is 30 second, the analyte is IgG in whole blood, the preferred separation distance is larger than 10 um, 17 um, 20 um, 30 um, 40 um, 50 um, 100 um.

In some embodiments, the intended assay time is 60 second, the analyte is IgG in PBS, the preferred separation distance is larger than 30 um, 40 um, 49 um, 50 um, 80 um, 100 um.

In some embodiments, the intended assay time is 60 second, the analyte is IgG in whole blood, the preferred separation distance is larger than 20 um, 24 um, 30 um, 40 um, 50 um, 100 um.

In some embodiments, the intended assay time is 60 second, the analyte is sDNA in PBS, the preferred separation distance is larger than 80 um, 94 um, 100 um, 120 um, 150 um.

In some embodiments, the intended assay time is 60 second, the analyte is sDNA in whole blood, the preferred separation distance is larger than 30 um, 40 um, 49 um, 50 um, 80 um, 100 um.

In some embodiments, the intended assay time is 60 second, the analyte is small particle as 20 nm diameter in PBS, the preferred separation distance is larger than 30 um, 34 um, 40 um, 50 um, 80 um, 100 um.

In some embodiments, the intended assay time is 60 second, the analyte is small particle as 20 nm diameter in whole blood, the preferred separation distance is larger than 10 um, 17 um, 20 um, 30 um, 40 um, 50 um, 100 um.

In some embodiments, the intended assay time is 60 second, the analyte is medium particle as 100 nm diameter in PBS, the preferred separation distance is larger than 10 um, 15 um, 30 um, 50 um.

In some embodiments, the intended assay time is 60 second, the analyte is medium particle as 100 nm diameter in whole blood, the preferred separation distance is larger than 5 um, 7.7 um, 10 um, 20 um, 30 um, 40 um, 50 um.

In some embodiments, the intended assay time is 60 second, the analyte is large particle as 1 um diameter in PBS, the preferred separation distance is larger than 3 um, 5 um, 10 um, 15 um, 30 um.

In some embodiments, the intended assay time is 60 second, the analyte is large particle as 1 um diameter in whole blood, the preferred separation distance is larger than 2 um, 2.4 um, 5 um, 10 um, 15 um, 30 um.

In some embodiments, “separation distance” can refer to a distance between two neighboring storage sites.

In some embodiments, “separation distance” can refer to a distance between two neighboring field of view (FoV) for detecting signal, wherein the field of view is not required to align with the storage site.

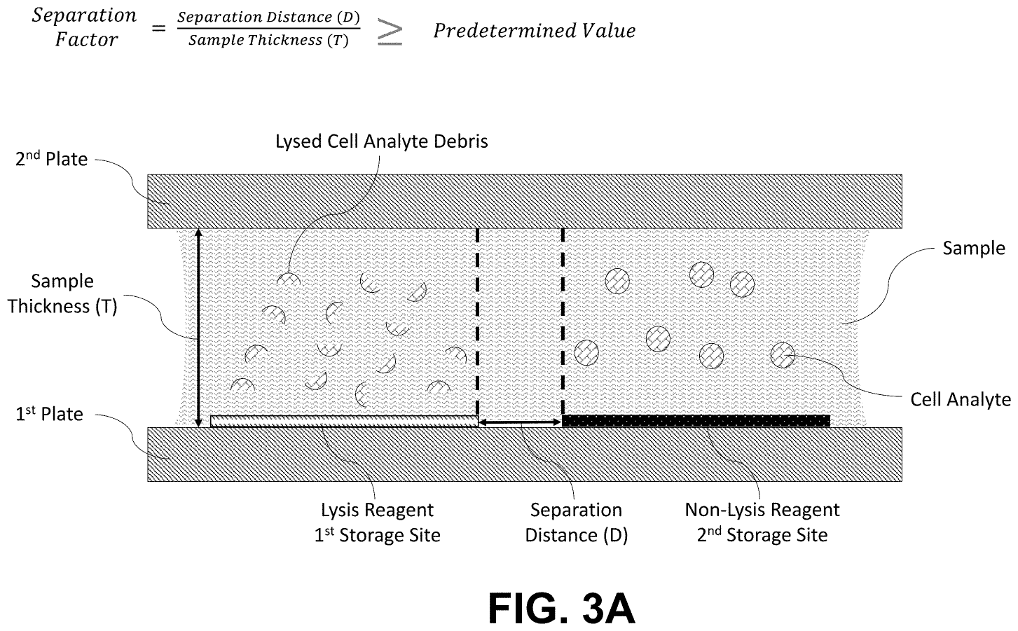

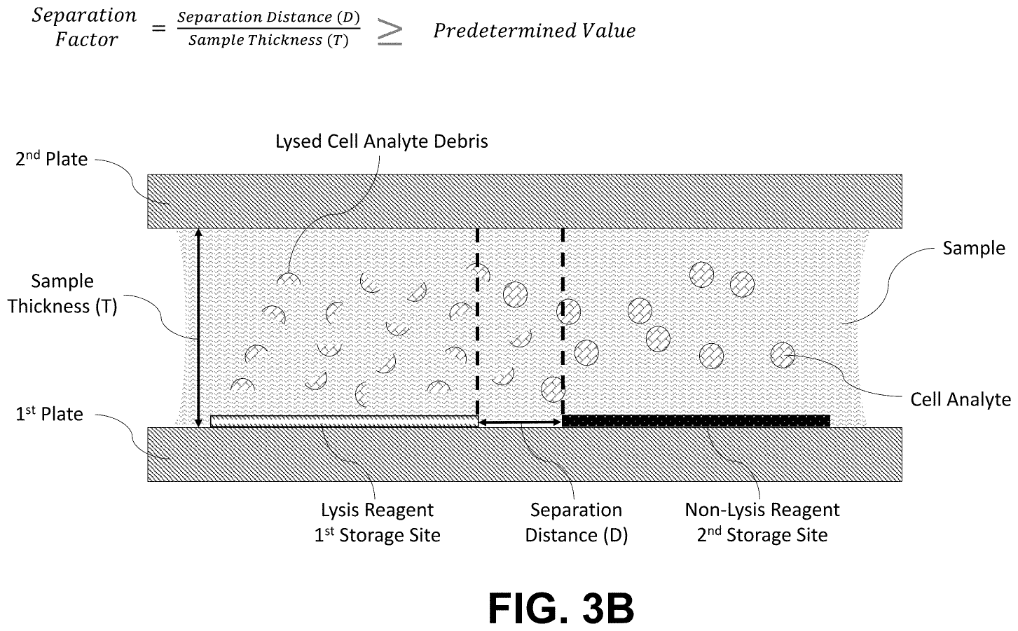

As illustrated in FIG. 3A, when the separation distance (D) is greater than a product of the separation factor and the sample thickness (T), reagents and/or reaction products from the first storage site do not mix or interfere with the reagents and/or reaction products of the second storage site. For example, given a sufficiently large separation distance, a first storage site comprising a lysis reagent, and the second storage site comprising a non-lysis reagent, the lysed cell analyte debris adjacent the first storage site does not mix with the lysed cell analyte debris of the second storage site. This allows for the independent analysis of each of the lysed cell analyte debris and the cell analytes. This is advantageous as it not only enables a user to perform assay simultaneously, but assays that cannot otherwise typically be compatible (e.g., a lysis assay and a non-lysis assay).

As illustrated in FIG. 3B, when the separation distance (D) is greater than a product of the separation factor and the sample thickness (T), reagents and/or reaction products from the first storage site do not contact the second storage site, and/or reagents and/or reaction products from the second storage site do not contact the first storage site. For example, given a sufficiently large separation distance, a first storage site comprising a lysis reagent, and the second storage site comprising a non-lysis reagent, the lysed cell analyte debris adjacent the first storage site does not contact the second storage site. This allows for the independent analysis of each of the lysed cell analyte debris and the cell analytes. This is advantageous as it not only enables a user to perform assay simultaneously, but assays that cannot otherwise typically be compatible (e.g. a lysis assay and a non-lysis assay).

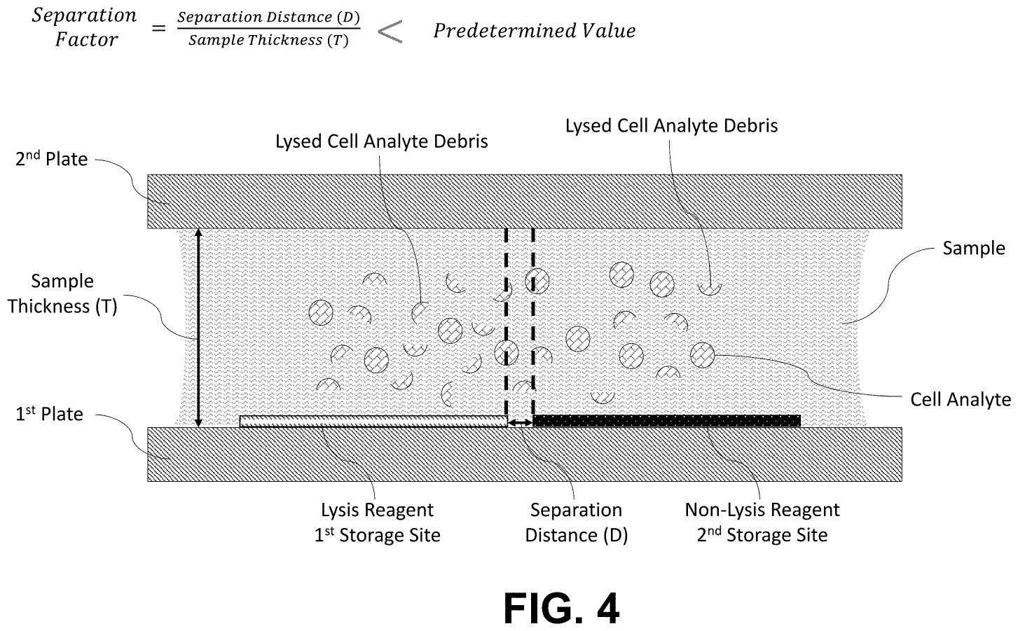

As illustrated in FIG. 4, when the separation distance (D) is less than a product of the separation factor and the sample thickness (T), reagents and/or reaction products from the first storage site can mix or interfere with the second storage site. For example, given an insufficiently large separation distance, a first storage site comprising a lysis reagent, and the second storage site comprising a non-lysis reagent, the lysed cell analyte debris adjacent the first storage site mixes with the lysed cell analyte debris of the second storage site. Independent analysis of each of the lysed cell analyte debris and the cell analytes is no longer possible.

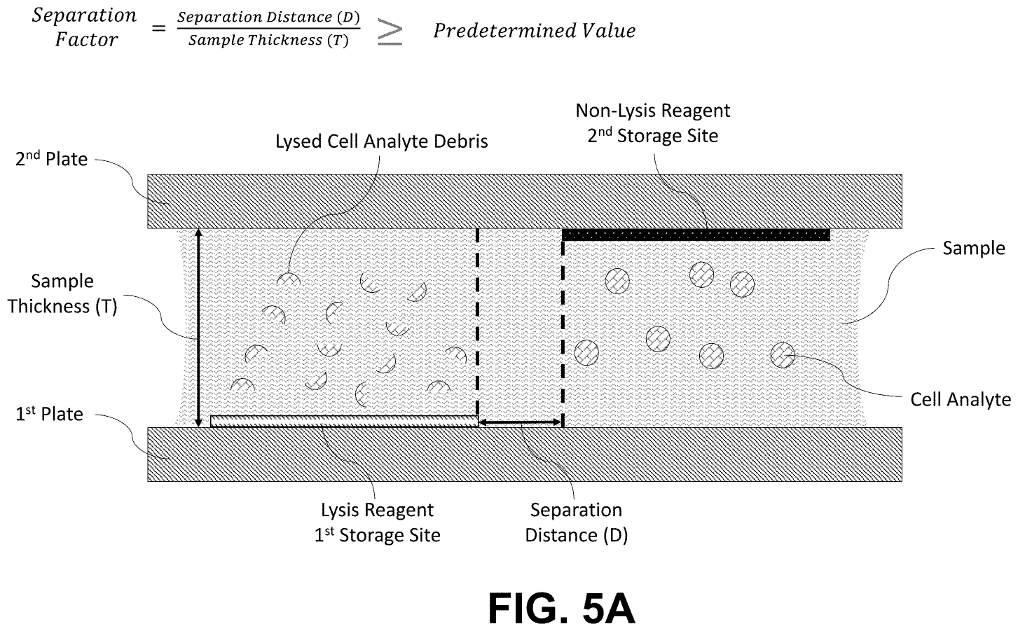

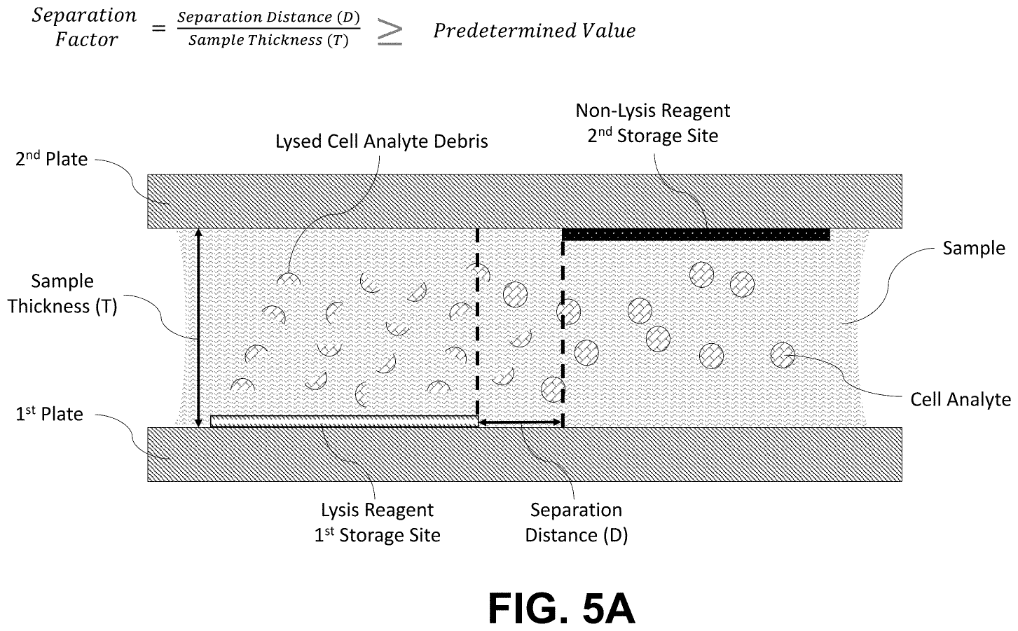

As illustrated in FIG. 5A, when the separation distance (D) is greater than a product of the separation factor and the sample thickness (T), reagents and/or reaction products from the first storage site do not mix or interfere with the reagents and/or reaction products of the second storage site. For example, given a sufficiently large separation distance, a first storage site on the first plate and comprising a lysis reagent, and the second storage site one the second plate and comprising a non-lysis reagent, the lysed cell analyte debris adjacent the first storage site does not mix with the lysed cell analyte debris of the second storage site. This allows for the independent analysis of each of the lysed cell analyte debris and the cell analytes. This is advantageous as it not only enables a user to perform assay simultaneously, but assays that cannot otherwise typically be compatible (e.g., a lysis assay and a non-lysis assay).

As illustrated in FIG. 5B, when the separation distance (D) is greater than a product of the separation factor and the sample thickness (T), reagents and/or reaction products from the first storage site do not contact the second storage site, and/or reagents and/or reaction products from the second storage site do not contact the first storage site. For example, given a sufficiently large separation distance, a first storage site on a first plate and comprising a lysis reagent, and the second storage site on the second plate and comprising a non-lysis reagent, the lysed cell analyte debris adjacent the first storage site does not contact the second storage site. This allows for the independent analysis of each of the lysed cell analyte debris and the cell analytes. This is advantageous as it not only enables a user to perform assay simultaneously, but assays that cannot otherwise typically be compatible (e.g., a lysis assay and a non-lysis assay).

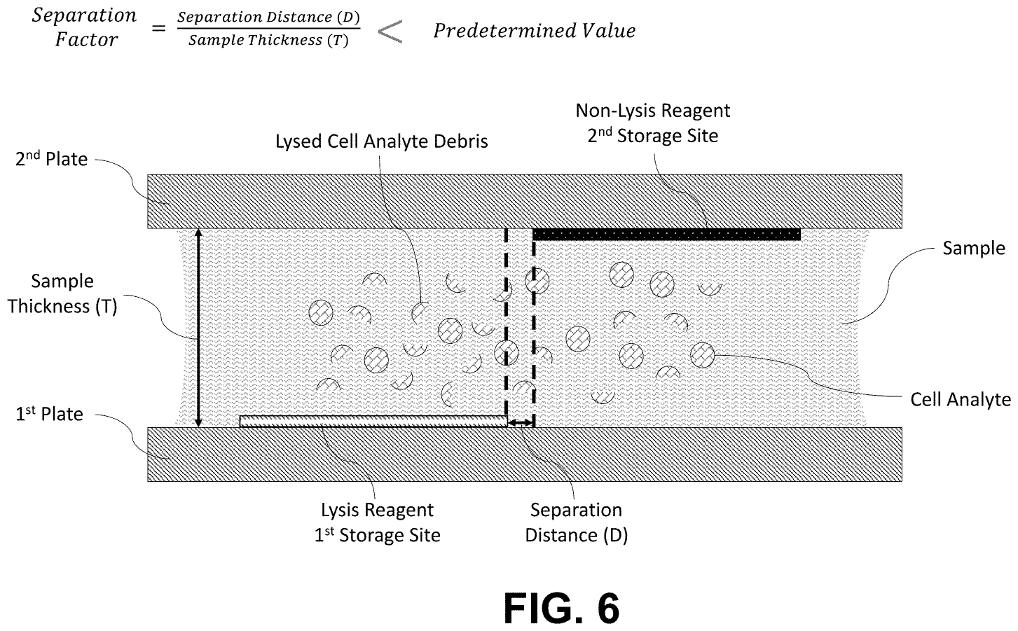

As illustrated in FIG. 6, when the separation distance (D) is less than a product of the separation factor and the sample thickness (T), reagents and/or reaction products from the first storage site mix or interfere with the second storage site. For example, given an insufficiently large separation distance, a first storage site on the first plate and comprising a lysis reagent, and the second storage site on a second plate and comprising a non-lysis reagent, the lysed cell analyte debris adjacent the first storage site mixes with the lysed cell analyte debris of the second storage site. Independent analysis of each of the lysed cell analyte debris and the cell analytes is no longer be possible.

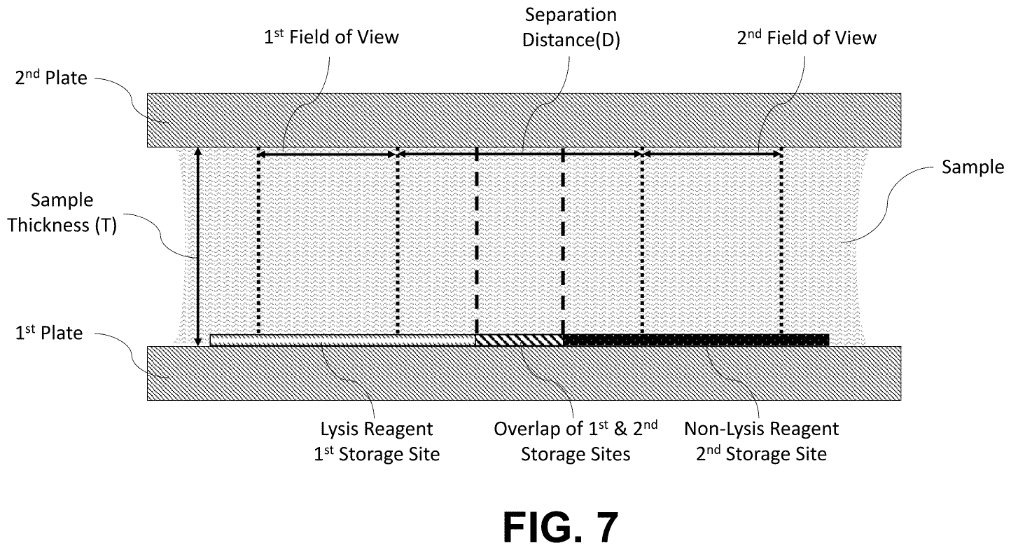

As illustrated in FIG. 7, in certain embodiments a device of the present disclosure can comprise two storage sites where at least a portion of each storage site overlap. When the separation distance (D), which in this embodiment is the distance between a first field of view within the first storage site and a second field of view within a second storage site, is greater than a product of the separation factor and the sample thickness (T), reagents and/or reaction products from the first storage site do not enter the second field of view. Similarly, when the separation distance (D) is greater than a product of the separation factor and the sample thickness (T), reagents and/or reaction products from the second storage site do not enter the first field of view. This allows for the independent analysis of each of the lysed cell analyte debris and the cell analytes. This is advantageous as it not only enables a user to perform assay simultaneously, but assays that cannot otherwise typically be compatible (e.g., a lysis assay and a non-lysis assay).

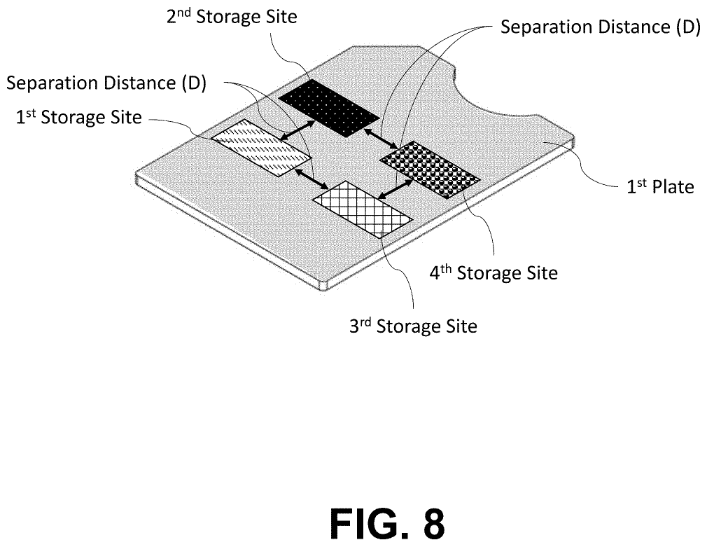

A QMAX card can have a plurality of storage sites. As illustrated in FIG. 8, a plate (e.g., a first plate) of a QMAX card can have four storage sites (each storage site having one or more reagents and/or capture agents) disposed thereon and separated by a separation distance (D). In certain embodiments, a storage site can be separated from two or more other storage sites by the same separation distance. In other embodiments, a storage site can be separated from two or more other storage sites by different separation distances. For example, the separation factor between a first storage site and a second storage site can be different from a separation factor between the first storage site and a third storage site.

Storage sites. The term “storage site” can refer to a site of an area on a plate, wherein the site contains reagents to be added into a sample, and the reagents are capable of being dissolved into the sample that is in contact with the reagents. In certain embodiments, a device of the present disclosure can have at least 2 storage sites. In certain embodiments, a device of the present disclosure can have a plurality of storage sites, for example, 2, 3, 4, 5 storage sites, 6, 7, 8, 9, 10, 20, 30, 40, 50, 75, 100, 150, 200, 250, 500, 750, 1,000, 1,500, 2,000, 2,500, 5,000, 10,000, greater than 10,000 sites, including intermediate values and ranges. For example, a device of the present disclosure can comprise 3 storage sites. In another example, a device of the present disclosure can comprise 10 storage sites. In certain embodiments, a device of the present disclosure can have between 2 storage sites and 10,000 storage sites. For example, a device of the present disclosure can comprise between 2 storage sites and 50 storage sites. In another example, a device of the present disclosure can comprise between 2 storage sites and 10 storage sites. In another example, a device of the present disclosure can comprise between 2 storage sites and 5 storage sites.

In certain embodiments, the storage sites can be disposed on one plate. For example, a device of the present disclosure can comprise 5 storage sites, and all 5 storage sites can be disposed on a single plate. In other embodiments, the storage sites can be disposed on two plates. For example, a device of the present disclosure can comprise 5 storage sites, with 2 storage sites disposed on a first plate and 3 storage sites disposed on a second plate.

A storage site can be any shape or size. In certain embodiments, a storage site can be a circle, a triangle, a square, a rectangle, a pentagon, a hexagon, a heptagon, an octagon, a nonagon, a decagon, a hendecagon, a dodecagon, a hexadecagon, an icosagon, a star. and like geometries. In certain embodiments, the storage site can be a triangle, and the triangle can be an acute triangle, an equilateral triangle, an isosceles triangle, an obtuse triangle, a rational triangle, a right triangle, a 30-60-90 triangle, an isosceles right triangle, a Kepler triangle, or a scalene triangle. In certain embodiments, the shape of a storage site can be a quadrilateral (e.g., a diamond), a cyclic quadrilateral, a square, a kite, a parallelogram, a rhombus, a Lozeng, a rhomboid, a rectangle, a tangential quadrilateral, a trapezoid, a trapezium, or an isososceles trapezoid. In certain embodiments, the storage site can be a crescent, an ellipse, a lune, an oval, a Reuleauz polygon, a Reuleaux triangle, a lens shape, a vesica piscis, a salinon, a semicircle, a tomoe, a magatama, a triquetra, an asteroid, a deltoid super ellipse, a tomahawk, and like shapes.

In certain embodiments, a storage site can have an area of less than or equal to about the following values: 20 cm², 15 cm², 10 cm², 9 cm², 8 cm², 7 cm², 6 cm², 5 cm², 4 cm², 3 cm², 2.5 cm², 2 cm², 1.5 cm², 1 cm², 0.9 cm², 0.8 cm², 0.7 cm², 0.6 cm², 0.5 cm², 0.4 cm², 0.3 cm², 0.2 cm², or less than or equal to about 0.1 cm², including intermediate values and ranges.

A device having multiple storage sites, wherein at least two of the storage sites have different areas, can be useful. An area of a storage site can be determined based on a concentration of an analyte in the sample. In certain embodiments, a greater concentration of an analyte in a sample can necessitate a smaller storage site area. In certain embodiments, a lower concentration of an analyte in a sample can necessitate a smaller storage site area. In certain embodiments, a device of the present disclosure can have a plurality of storage sites, and at least two of the plurality of storage sites have different areas. For example, a device of the present disclosure can have a first storage site having an area of about 1 cm² for detecting white blood cells and a second storage site having an area of about 0.1 cm² for detecting red blood cells.

QMAX System

A. QMAX Card

Details of the QMAX card are described in detail in a variety of publications including International Application No. PCT/US2016/046437 (Essenlix Docket No. ESSN-028WO), which is incorporated by reference here in its entirety.

I. Plates

In present invention, generally, the plates of CROF are made of any material that: (i) is capable of being used to regulate, together with the spacers, the thickness of a portion or entire volume of the sample; and (ii) has no significant adverse effects to a sample, an assay, or a goal that the plates intend to accomplish. However, in certain embodiments, particular materials (hence their properties) can be used for the plate to achieve certain objectives.

In certain embodiments, the two plates can have the same or different parameters for each of the following parameters: plate material, plate thickness, plate shape, plate area, plate flexibility, plate surface property, and plate optical transparency.

(i) Plate Materials. The plates can be made of a single material, composite materials, multiple materials, multilayer of materials, alloys, or a combination thereof. Each of the materials for the plate is an inorganic material, an organic material, or a mixture thereof, wherein examples of the materials follow in Mat-1 and Mat-2.

Mat-1: The inorganic materials for the plates include, for example, glass, quartz, oxides, silicon-dioxide, silicon-nitride, hafnium oxide (HfO), aluminum oxide (AlO), semiconductors: (e.g., silicon, GaAs, GaN, etc.), metals (e.g., gold, silver, coper, aluminum, Ti, Ni, etc.), ceramics, or any combinations of thereof.

Mat-2: The organic materials for the spacers can include, for example, polymers (e.g., plastics) or amorphous organic materials. The polymer materials for the spacers can include, for example, acrylate, vinyl, olefin, cellulosic, noncellulosic, polyester, Nylon, cyclic olefin copolymer (COC), poly(methyl methacrylate) (PMMA), polycarbonate (PC), cyclic olefin polymer (COP), liquid crystalline polymer (LCP), polyimide (PA), polyethylene (PE), polyimide (PI), polypropylene (PP), poly(phenylene ether) (PPE), polystyrene (PS), polyoxymethylene (POM), polyether ether ketone (PEEK), polyether sulfone (PES), poly(ethylene phthalate) (PET), polytetrafluoroethylene (PTFE), polyvinyl chloride (PVC), polyvinylidene fluoride (PVDF), polybutylene terephthalate (PBT), fluorinated ethylene propylene (FEP), perfluoroalkoxyalkane (PFA), polydimethylsiloxane (PDMS), rubbers, or any combinations of thereof.

In certain embodiments, the plates can be each independently made of at least one of glass, plastic, ceramic, and metal. In certain embodiments, each plate independently includes at least one of glass, plastic, ceramic, and metal.

In certain embodiments, one plate can be different from the other plate in lateral area, thickness, shape, materials, or surface treatment. In certain embodiments, one plate can be the same as the other plate in lateral area, thickness, shape, materials, or surface treatment.

The materials for the plates are rigid, flexible or any flexibility between the two. The rigid (i.e., stiff) or flexibility is relative to a given pressing forces used in bringing the plates into the closed configuration.

In certain embodiments, a selection of rigid or flexible plate can be determined from the requirements of controlling a uniformity of the sample thickness at the closed configuration.

In certain embodiments, at least one of the two plates are transparent (e.g., to a light). In certain embodiments at least a part or several parts of one plate or both plates are transparent. In certain embodiments, the plates are non-transparent.

(ii) Plate Thickness. In certain embodiments, the average thicknesses for at least one of the pates can be, for example, 2 nm or less, 10 nm or less, 100 nm or less, 500 nm or less, 1000 nm or less, 2 um (micron) or less, 5 um or less, 10 um or less, 20 um or less, 50 um or less, 100 um or less, 150 um or less, 200 um or less, 300 um or less, 500 um or less, 800 um or less, 1 mm (millimeter) or less, 2 mm or less, 3 mm or less, including intermediate values and ranges.

In certain embodiments, the average thicknesses for at least one of the plates can be, for example, at most 3 mm (millimeter), at most 5 mm, at most 10 mm, at most 20 mm, at most 50 mm, at most 100 mm, at most 500 mm, including intermediate values and ranges.

In certain embodiments, the thickness of a plate is not uniform across the plate. Using a different plate thickness at a different location(s) can be used to control the plate bending, folding, sample thickness regulation, and other plate characteristics.

(iii) Plate Shape and Area. Generally, the plates can have any shapes, as long as the shape allows a compressed open flow of the sample and the regulation of the sample thickness. However, in certain embodiments, a particular shape can be advantageous. The shape of the plate can be, for example, round, elliptical, rectangles, triangles, polygons, ring-shaped, or any superpositions of these shapes.

In certain embodiments, the two plates can have the same size or shape, or different size or shape. The area of the plates depends on the application. The area of the plate is at most 1 mm² (millimeter square), at most 10 mm2, at most 100 mm2, at most 1 cm2 (centimeter square), at most 5 cm2, at most 10 cm2, at most 100 cm2, at most 500 cm2, at most 1000 cm2, at most 5000 cm2, at most 10,000 cm2, or over 10,000 cm2, including intermediate values and ranges. The shape of the plate can be rectangle, square, round, or shape others.

In certain embodiments, at least one of the plates is in the form of a belt (or strip) that has a width, thickness, and length. The width can be, for example, at most 0.1 cm (centimeter), at most 0.5 cm, at most 1 cm, at most 5 cm, at most 10 cm, at most 50 cm, at most 100 cm, at most 500 cm, at most 1000 cm, including intermediate values and ranges. The length can be as long it needed. The belt can be rolled into a roll.

(iv) Plate Surface Flatness. In many embodiments, an inner surface of the plates can be, for example, flat or significantly flat, or planar. In certain embodiments, the two inner surfaces can be, for example, at the closed configuration, parallel with each other. Flat inner surfaces facilitate a quantification and/or controlling of the sample thickness by simply using the predetermined spacer height at the closed configuration. For non-flat inner surfaces of the plate, one needs to know not only the spacer height, but also the exact the topology of the inner surface to quantify and/or control the sample thickness at the closed configuration. To know the surface topology one needs additional measurements and/or corrections, which can be complex, time consuming, and costly.

A flatness of the plate surface is relative to the final sample thickness (the final thickness is the thickness at the closed configuration), and is often characterized by the term of “relative surface flatness”, which is the ratio of the plate surface flatness variation to the final sample thickness.

In certain embodiments, the relative surface less than 0.01%, 0.1%, less than 0.5%, less than 1%, less than 2%, less than 5%, less than 10%, less than 20%, less than 30%, less than 50%, less than 70%, less than 80%, less than 100%, including intermediate values and ranges.

(v) Plate Surface Parallelness. In certain embodiments, the two surfaces of the plate can be, for example, significantly parallel with each other. In certain embodiments, the two surfaces of the plate can be, for example, not parallel with each other.

(vi) Plate Flexibility. In certain embodiments, a plate is flexible under the compressing of a CROF process. In certain embodiments, both plates are flexible under the compressing of a CROF process. In certain embodiments, a plate is rigid and another plate is flexible under the compressing of a CROF process. In certain embodiments, both plates are rigid. In certain embodiments, both plate are flexible but have different flexibility.

(vii) Plate Optical Transparency. In certain embodiments, a plate is optical transparent. In certain embodiments, both plates are optical transparent. In certain embodiments, a plate is optical transparent and another plate is opaque. In certain embodiments, both plates are opaque. In certain embodiments, both plates can be, for example, optically transparent but have different optical transparency. The optical transparency of a plate can refer to a part or the entire area of the plate.

(viii) Surface Wetting Properties. In certain embodiments, a plate has an inner surface that wets (i.e., contact angle is less 90 degree) the sample, the transfer liquid, or both. In certain embodiments, both plates have an inner surface that wets the sample, the transfer liquid, or both; either with the same or different wettability. In certain embodiments, a plate has an inner surface that wets the sample, the transfer liquid, or both; and another plate has an inner surface that does not wet (i.e., the contact angle equal to or larger than 90 degree). The wetting of a plate inner surface can refer to a part or the entire area of the plate.

In certain embodiments, the inner surface of the plate has other nano or microstructures to control a lateral flow of a sample during a CROF. The nano- or microstructures include, but not limited to, channels, pumps, and others. Nano- and microstructures are also used to control the wetting properties of an inner surface.

II. Spacers

(i) Spacers' Function. In present invention, the spacers are configured to have one or any combinations of the following functions and properties, the spacers are configured to: (1) control, together with the plates, the thickness of the sample or a relevant volume of the sample (Preferably, the thickness control is precise, or uniform or both, over a relevant area); (2) allow the sample to have a compressed regulated open flow (CROF) on plate surface; (3) not take significant surface area (volume) in a given sample area (volume); (4) reduce or increase the effect of sedimentation of particles or analytes in the sample; (5) change and/or control the wetting propertied of the inner surface of the plates; (6) identify a location of the plate, a scale of size, and/or the information related to a plate, or (7) do any combination of the above.

(ii) Spacer Architectures and Shapes. To achieve desired sample thickness reduction and control, in certain embodiments, the spacers are fixed its respective plate. In general, the spacer can have any shape, as long as the spacers are capable of regulating the sample thickness during a CROF process, but certain shapes are preferred to achieve certain functions, such as better uniformity, less overshoot in pressing, and like considerations.

The spacer(s) is a single spacer or a plurality of spacers. (e.g., an array). Certain embodiments of a plurality of spacers is an array of spacers (e.g., pillars), where the inter-spacer distance is periodic or aperiodic, or is periodic or aperiodic in certain areas of the plates, or has different distances in different areas of the plates.

There are two kinds of the spacers: open-spacers and enclosed-spacers. An open-spacer is the spacer that allows a sample to flow through the spacer (i.e., the sample flows around and past the spacer, for example, a post as the spacer), and an enclosed spacer is a spacer that stops the sample flow (i.e., the sample cannot flow beyond the spacer, for example, a ring shape spacer and the sample is inside the ring). Both types of spacers use their height to regulate the final sample thickness at a closed configuration.

In certain embodiments, the spacers are open-spacers only. In certain embodiments, the spacers are enclosed-spacers only. In certain embodiments, the spacers are a combination of open-spacers and enclosed-spacers.

The term “pillar spacer” means that the spacer has a pillar shape and the pillar shape can refer to an object that has height and a lateral shape that allow a sample to flow around it during a compressed open flow.

In certain embodiments, the lateral shapes of the pillar spacers are the shape selected from: (i) round, elliptical, rectangles, triangles, polygons, ring-shaped, star-shaped, letter-shaped (e.g., L-shaped, C-shaped, i.e., the letters from A to Z), number shaped (e.g., the shapes like 0 1, 2, 3, 4, . . . to 9); (ii) the shapes in group (i) with at least one rounded corner; (iii) the shape from group (i) with zig-zag or rough edges; and (iv) any superposition of any of (i), (ii), and (iii). For multiple spacers, different spacers can have a different lateral shape and size, and a different distance from the neighboring spacers.

In certain embodiments, the cross-sectional shape of the pillars or spacers can be, for example, a circle, triangle, square, rectangle, pentagon, hexagon, heptagon, octagon, nonagon, decagon, hendecagon, dodecagon, hexadecagon, icosagon, or star. In certain embodiments, a triangle can be an acute triangle, equilateral triangle, isosceles triangle, obtuse triangle, rational triangle, right triangle (e.g., 30-60-90 triangle, isosceles right triangle, Kepler triangle), or scalene triangle. In certain embodiments, the cross-sectional shape of a pillar or spacer can be a quadrilateral, a diamond, cyclic quadrilateral, square, kite, parallelogram, rhombus, Lozeng, rhomboid, rectangle, tangential quadrilateral, trapezoid, trapezium, or isososceles trapezoid. In certain embodiments, the cross-sectional shape of a pillar or spacer can be a crescent, ellipse, lune, oval, Reuleauz polygon, Reuleaux triangle, lens, vesica piscis, salinon, semicircle, tomoe, magatama, triquetra, asteroid, deltoid super ellipse, or tomahawk. In certain embodiments, a cross- sectional shape with a point has a sharpened point. In some cases, a cross-sectional shape with a point has a rounded point. In some cases, a cross-sectional shape with more than one point has all rounded points, all sharpened points or at least one rounded point and at least one sharpened point. In certain embodiments, a pillar or spacer can have a cylindrical shape. In certain embodiments, each pillar or spacer in a plurality of pillars or spacers can be the same, e.g., having the same shape and size. In certain embodiments, each pillar or spacer in a plurality of pillars or spacers can have different shapes and/or sizes.

In certain embodiments, the spacers can be and/or can include posts, columns, beads, spheres, and/or other suitable geometries. The lateral shape and dimension (i.e., transverse to the respective plate surface) of the spacers can be anything, except, in certain embodiments, the following restrictions: (i) the spacer geometry will not cause a significant error in measuring the sample thickness and volume; or (ii) the spacer geometry would not prevent the out-flowing of the sample between the plates (i.e., it is not in enclosed form). But in certain embodiments, they require some spacers to be closed spacers to restrict the sample flow.

In certain embodiments, the shapes of the spacers have rounded corners. For example, a rectangle shaped spacer has one, several, or all corners rounded (like a circle rather than a 90 degree angle). A round corner often make a fabrication of the spacer easier, and in some cases less damage to a biological material.

The sidewall of the pillars can be straight, curved, sloped, or different shaped in different section of the sidewall. In certain embodiments, the spacers are pillars of various lateral shapes, sidewalls, and pillar-height to pillar lateral area ratio. In a preferred embodiment, the spacers have shapes of pillars for allowing open flow.

(iii) Spacers' Materials. In the present invention, the spacers are generally made of any material that is capable of being used to regulate, together with the two plates, the thickness of a relevant volume of the sample. In certain embodiments, the materials for the spacers are different from that for the plates. In certain embodiments, the materials for the spaces are at least the same as a part of the materials for at least one plate.

The spacers are made a single material, composite materials, multiple materials, multilayer of materials, alloys, or a combination thereof. Each of the materials for the spacers is an inorganic material, an organic material, or a mixture thereof, wherein examples of the materials are given in the abovementioned Mat-1 and Mat-2 sections. In a preferred embodiment, the spacers are made in the same material as a plate used in CROF.

(iv) Spacers' Mechanical Strength and Flexibility. In certain embodiments, the mechanical strength of the spacers are strong enough, so that during the compression and at the closed configuration of the plates, the height of the spacers is the same or significantly same as that when the plates are in an open configuration. In certain embodiments, the differences of the spacers between the open configuration and the closed configuration can be characterized and predetermined.

The material for the spacers can be, for example, rigid, flexible, or any flexibility between the two. The rigid is relative to a given pressing force used in bringing the plates into the closed configuration: if the space does not deform greater than 1% in its height under the pressing force, the spacer material is regarded as rigid, otherwise a flexible spacer material. When a spacer is made of flexible material, the final sample thickness at a closed configuration still can be predetermined from the pressing force and the mechanical property of the spacer.

(v) Spacers Inside Sample. To achieve desired sample thickness reduction and control, particularly to achieve a good sample thickness uniformity, in certain embodiments, the spacers are placed inside the sample, or the relevant volume of the sample. In certain embodiments, there are one or more spacers inside the sample or the relevant volume of the sample, with a proper inter spacer distance. In certain embodiments, at least one of the spacers is inside the sample area, at least two of the spacers inside the sample area or the relevant volume of the sample, or at least “n” spacers inside the sample area or the relevant volume of the sample, where “n” can be determined by a sample thickness uniformity or a required sample flow property during a CROF.

(vi) Spacer Height. In certain embodiments, all spacers have the same pre-determined height. In certain embodiments, spacers have different pre-determined height. In certain embodiments, spacers can be divided into groups or regions, wherein each group or region has its own spacer height. And in certain embodiments, the predetermined height of the spacers is an average height of the spacers. In certain embodiments, the spacers have approximately the same height. In certain embodiments, a percentage of a number of the spacers have the same height.

The height of the spacers is selected by a desired regulated final sample thickness and the residue sample thickness. The spacer height (the predetermined spacer height) and/or sample thickness can be, for example, 3 nm or less, 10 nm or less, 50 nm or less, 100 nm or less, 200 nm or less, 500 nm or less, 800 nm or less, 1000 nm or less, 1 um or less, 2 um or less, 3 um or less, 5 um or less, 10 um or less, 20 um or less, 30 um or less, 50 um or less, 100 um or less, 150 um or less, 200 um or less, 300 um or less, 500 um or less, 800 um or less, 1 mm or less, 2 mm or less, 4 mm or less, including intermediate values and ranges.

The spacer height and/or sample thickness is between 1 nm to 100 nm in one preferred embodiment, 100 nm to 500 nm in another preferred embodiment, 500 nm to 1000 nm in a separate preferred embodiment, 1 um (i.e., 1000 nm) to 2 um in another preferred embodiment, 2 um to 3 um in a separate preferred embodiment, 3 um to 5 um in another preferred embodiment, 5 um to 10 um in a separate preferred embodiment, and 10 um to 50 um in another preferred embodiment, 50 um to 100 um in a separate preferred embodiment.

In certain embodiments, the spacer height and/or sample thickness (i) equal to or slightly larger than the minimum dimension of an analyte, or (ii) equal to or slightly larger than the maximum dimension of an analyte. The “slightly larger” means that it is about 1% to 5% larger and any number between the two values.

In certain embodiments, the spacer height and/or sample thickness is larger than the minimum dimension of an analyte (e.g., an analyte has an anisotropic shape), but less than the maximum dimension of the analyte.

For example, the red blood cell has a disk shape with a minimum dimension of 2 um (disk thickness) and a maximum dimension of 11 um (a disk diameter). In an embodiment of the present invention, the spacers is selected to make the inner surface spacing of the plates in a relevant area to be 2 um (equal to the minimum dimension) in one embodiment, 2.2 um in another embodiment, or 3 (50% larger than the minimum dimension) in other embodiment, but less than the maximum dimension of the red blood cell. Such embodiment has certain advantages in blood cell counting. In one embodiment, for red blood cell counting, by making the inner surface spacing at 2 or 3 um and any number between the two values, a undiluted whole blood sample is confined in the spacing, on average, each red blood cell (RBC) does not overlap with others, allowing an accurate counting of the red blood cells visually. Too many overlaps between the RBC's can cause serious errors in counting.

In certain embodiments, the present invention uses the plates and the spacers to regulate not only a thickness of a sample, but also the orientation and/or surface density of the analytes/entity in the sample when the plates are at the closed configuration. When the plates are at a closed configuration, a thinner thickness of the sample gives less analytes/entity per surface area (i.e., less surface concentration).

(vii) Spacer Lateral Dimension. For an open-spacer, the lateral dimensions can be characterized by its lateral dimension (also referred to as width) in the x and y—two orthogonal directions. The lateral dimension of a spacer in each direction can be, for example, the same or different.

In certain embodiments, the ratio of the lateral dimensions of x to y direction can be, for example, 1, 1.5, 2, 5, 10, 100, 500, 1000, 10,000, including intermediate values and ranges. In certain embodiments, a different ratio can be, for example, used to regulate the sample flow direction; the larger the ratio, the flow is along one direction (larger size direction).

In certain embodiments, the different lateral dimensions of the spacers in the x and y direction are used as: (a) using the spacers as scale-markers to indicate the orientation of the plates; (b) using the spacers to create more sample flow in a preferred direction; or both.

In a preferred embodiment, the period, width, and height have the same dimension.

In certain embodiments, all spacers have the same shape and dimensions. In certain embodiments, each of the spacers have different lateral dimensions.

For enclosed-spacers, in certain embodiments, the inner lateral shape and size are selected based on the total volume of a sample to be enclosed by the enclosed spacer(s), wherein the volume size has been described in the present disclosure; and in certain embodiments, the outer lateral shape and size are selected based on the needed strength to support the pressure of the liquid against the spacer and the compress pressure that presses the plates together.

(viii) Aspect Ratio of Height to the Average Lateral Dimension of Pillar Spacer. In certain embodiments, the aspect ratio of the height to the average lateral dimension of the pillar spacer is 100,000, 10,000, 1,000, 100, 10, 1, 0.1, 0.01, 0.001, 0.0001, 0, 00001, including intermediate values and ranges.

(ix) Spacer Height Precisions. The spacer height should be controlled precisely. The relative precision of the spacer (i.e., the ratio of the deviation to the desired spacer height) is 0.001% or less, 0.01% or less, 0.1% or less; 0.5% or less, 1% or less, 2% or less, 5% or less, 8% or less, 10% or less, 15% or less, 20% or less, 30% or less, 40% or less, 50% or less, 60% or less, 70% or less, 80% or less, 90% or less, 99.9% or less, .

(x) Inter-Spacer Distance. The spacers can be a single spacer or a plurality of spacers on the plate or in a relevant area of the sample. In certain embodiments, the spacers on the plates are configured and/or arranged in an array form, and the array is a periodic, non-periodic array or periodic in some locations of the plate and non-periodic in other locations.

In certain embodiments, the periodic array of the spacers has a spacer lattice shape of square, rectangle, triangle, hexagon, polygon, or any combinations of thereof, where a combination means that different locations of a plate has different spacer lattices.

In certain embodiments, the inter-spacer distance of a spacer array can be, for example, periodic (i.e., uniform inter-spacer distance) in at least one direction of the array. In certain embodiments, the inter-spacer distance is configured to improve the uniformity between the plate spacing at a closed configuration.

The distance between neighboring spacers (i.e., the inter-spacer distance) can be, for example, 1 um or less, 5 um or less, 10 um or less, 20 um or less, 30 um or less, 40 um or less, 50 um or less, 60 um or less, 70 um or less, 80 um or less, 90 um or less, 100 um or less, 200 um or less, 300 um or less, 400 um or less, including intermediate values and ranges.

In certain embodiments, the inter-spacer distance can be, for example, at 400 or less, 500 or less, 1 mm or less, 2 mm or less, 3 mm or less, 5mm or less, 7 mm or less, 10 mm or less, or any range between the values. In certain embodiments, the inter-spacer distance can be, for example, 10 mm or less, 20 mm or less, 30 mm or less, 50 mm or less, 70 mm or less, 100 mm or less, including intermediate values and ranges.

The distance between neighboring spacers (i.e., the inter-spacer distance) is selected so that for a given properties of the plates and a sample, at the closed-configuration of the plates, the sample thickness variation between two neighboring spacers is, in certain embodiments, at most 0.5%, 1%, 5%, 10%, 20%, 30%, 50%, or 80%, including intermediate values and ranges; or in certain embodiments, at most 80%, 100%, 200%, or 400%, including intermediate values and ranges.

Clearly, for maintaining a given sample thickness variation between two neighboring spacers, when a more flexible plate is used, a closer inter-spacer distance is needed.

In a preferred embodiment, the spacer is a periodic square array, wherein the spacer is a pillar that has a height of 2 to 4 um, an average lateral dimension of from 5 to 20 um, and inter-spacer spacing of 1 um to 100 um.

In a preferred embodiment, the spacer is a periodic square array, wherein the spacer is a pillar that has a height of 2 to 4 um, an average lateral dimension of from 5 to 20 um, and inter-spacer spacing of 100 um to 250 um.

In a preferred embodiment, the spacer is a periodic square array, wherein the spacer is a pillar that has a height of 4 to 50 um, an average lateral dimension of from 5 to 20 um, and inter-spacer spacing of 1 um to 100 um.

In a preferred embodiment, the spacer is a periodic square array, wherein the spacer is a pillar that has a height of 4 to 50 um, an average lateral dimension of from 5 to 20 um, and inter-spacer spacing of 100 um to 250 um.

The period of spacer array is between 1 nm to 100 nm in one preferred embodiment, 100 nm to 500 nm in another preferred embodiment, 500 nm to 1000 nm in a separate preferred embodiment, 1 um (i.e., 1000 nm) to 2 um in another preferred embodiment, 2 um to 3 um in a separate preferred embodiment, 3 um to 5 um in another preferred embodiment, 5 um to 10 um in a separate preferred embodiment, and 10 um to 50 um in another preferred embodiment, 50 um to 100 um in a separate preferred embodiment, 100 um to 175 um in a separate preferred embodiment, and 175 um to 300 um in a separate preferred embodiment.

(xi) Spacer Density. The spacers are arranged on the respective plates at a surface density of greater than one per: 1 um², 10 um², 100 um², 500 um², 1000 um², 5,000 um², 0.01 mm², 0.1 mm², 1 mm², 5 mm², 10 mm², 100 mm², 1,000 mm², 10,000 mm², including intermediate values and ranges.

In some embodiments, the spacers are configured to occupy an insignificant surface area (volume) in a given sample area (volume).

(xii) Ratio of Spacer Volume to Sample Volume. In many embodiments, the ratio of the spacer volume (i.e., the volume of the spacer(s)) to sample volume (i.e., the volume of the sample), and/or the ratio of the volume of the spacers that are inside of the relevant volume of the sample, to the relevant volume of the sample are controlled for achieving certain advantages. The advantages include, for example, the uniformity of the sample thickness control, the uniformity of analytes, the sample flow properties (i.e., flow speed, flow direction, and like considerations).

In certain embodiments, the ratio of the spacer volume (r) to sample volume (v), and/or the ratio of the volume of the spacers that are inside of the relevant volume of the sample, to the relevant volume of the sample is less than 100%, at most 99%, at most 70%, at most 50%, at most 30%, at most 10%, at most 5%, at most 3% at most 1%, at most 0.1%, at most 0.01%, at most 0.001%, including intermediate values and ranges.

(xiii) Spacers Fixed to Plates. The inter spacer distance and the orientation of the spacers, which play a key role in the present invention, are preferably maintained during the process of bringing the plates from an open configuration to the closed configuration, and/or are preferably predetermined before the process from an open configuration to a closed configuration.

In certain embodiments of the present disclosure, spacers are fixed on one of the plates before bring the plates to the closed configuration. The term “a spacer is fixed with its respective plate” means that the spacer is attached to a plate and the attachment is maintained during a use of the plate. An example of “a spacer is fixed with its respective plate” is that a spacer is monolithically made of one piece of material of the plate, and the position of the spacer relative to the plate surface does not change. An example of “a spacer is not fixed with its respective plate” is that a spacer is glued to a plate by an adhesive, but during a use of the plate, the adhesive cannot hold the spacer at its original location on the plate surface (i.e., the spacer moves away from its original position on the plate surface).

In certain embodiments, at least one of the spacers are fixed to its respective plate. In certain embodiments, at two spacers are fixed to its respective plates. In certain embodiments, a majority of the spacers are fixed with their respective plates. In certain embodiments, all of the spacers are fixed with their respective plates.

In certain embodiments, a spacer is fixed to a plate monolithically.

In certain embodiments, the spacers are fixed to its respective plate by one or any combination of the following methods and/or configurations: attached to, bonded to, fused to, imprinted, and etched.

The term “imprinted” means that a spacer and a plate are fixed monolithically by imprinting (i.e., embossing) a piece of a material to form the spacer on the plate surface. The material can be a single layer or multiple layers.

The term “etched” means that a spacer and a plate are fixed monolithically by etching a piece of a material to form the spacer on the plate surface. The material can be a single layer or multiple layers.

The term “fused to” means that a spacer and a plate are fixed monolithically by attaching a spacer and a plate together, the original materials for the spacer and the plate fused to each other, and there is clear material boundary between the two materials after the fusion.

The term “bonded to” means that a spacer and a plate are fixed monolithically by binding a spacer and a plate by adhesion, for example, with a suitable adhesive.

The term “attached to” means that a spacer and a plate are connected together.

In certain embodiments, the spacers and the plate are made of the same materials. In other embodiment, the spacers and the plate are made of different materials. In other embodiment, the spacer and the plate are formed in one piece. In other embodiment, the spacer has one end fixed to its respective plate, while the other end is open for accommodating different configurations of the two plates.

In other embodiment, each of the spacers is independently at least one of: attached to, bonded to, fused to, imprinted in, or etched in the respective plate. The term “independently” means that one spacer is fixed with its respective plate by a same or a different method that is selected from the recited methods.

In certain embodiments, at least a distance between two spacers is predetermined. “Predetermined inter-spacer distance” means that the distance is known when a user uses the plates.

In certain embodiments of all methods and devices described herein, there can be additional or alternative spacer configurations besides fixed spacers.

(xiv) Specific Sample Thickness. In the present invention, it was observed that a larger plate holding force (i.e., the force that holds the two plates together) can be achieved by using a smaller plate spacing (for a given sample area), or a larger sample area (for a given plate-spacing), or both.

In certain embodiments, at least one of the plates is transparent in a region encompassing the relevant area, and each plate has an inner surface configured to: contact the sample in the closed configuration; the inner surfaces of the plates are substantially parallel with each other, in the closed configuration; the inner surfaces of the plates are substantially planar, except the locations that have the spacers; or any combination of thereof.

The spacers can be fabricated on a plate in a variety of ways, using lithography, etching, embossing (e.g., nanoimprinting), depositions, lift-off, fusing, or a combination of thereof. In certain embodiments, the spacers are directly embossed or imprinted on the plates. In certain embodiments, the spacers can be imprinted into a material (e.g., a plastic) that is deposited on the plates. In certain embodiments, the spacers can be made by directly embossing a surface of a CROF plate. The nanoimprinting can be done by roll-to-roll technology using a roller imprinter, or a roll-to-a-planar nanoimprint. Such processes can have a great economic advantage and lower manufacturing cost.

In certain embodiments, the spacers can be, for example, deposited on the plates. The deposition can be evaporation, pasting, or a lift-off. In pasting, the spacer is fabricated first on a carrier, then the spacer is transferred from the carrier to the plate. In the lift-off, a removable material is first deposited on the plate and holes are created in the material; the hole bottom exposes the plate surface and then a spacer material is deposited into the hole and afterwards the removable material is removed, leaving only the spacers on the plate surface. In certain embodiments, the spacers deposited on the plate are fused with the plate. In certain embodiments, the spacer and the plates are fabricated in a single process. The single process includes imprinting (i.e., embossing, molding) or synthesis.

In certain embodiments, at least two of the spacers are fixed to the respective plate by different fabrication methods, and optionally wherein the different fabrication methods include at least one of, for example: deposition, bonded, fuse, imprinted, and etched.

In certain embodiments, one or more of the spacers are fixed to the respective plate(s) by a fabrication method of, for example: bonded, fused, imprinted, or etched, or any combination of thereof.

In certain embodiments, the fabrication methods for forming such monolithic spacers on the plate include a method of, for example: bonded, fused, imprinted, or etched, or any combination of thereof.

B. Adaptor

Details of the adaptor are described in a variety of publications including International Application No. PCT/US2018/017504, which is incorporated by reference for all purposes.

In some embodiments, the present invention provides a system comprising an optical adaptor and a smartphone. The optical adaptor device fits over a smartphone converting it into a microscope which can take both fluorescent and bright-field images of a sample. This system can be operated conveniently and reliably by a common person at any location. The optical adaptor takes advantage of the existing resources of the smartphone, including camera, light source, memory, processor and display screen, which provides a low-cost solution for the user to do bright-field and fluorescent microscopy.

In this invention, the optical adaptor device comprises a holder frame fitting over the upper part of the smartphone and an optical box attached to the holder having sample receptacle slot and illumination optics. In some references (e.g., U.S. Pat. Nos. 2016/029091 and 2011/0292198), the optical adaptor design is a whole piece including both clip-on mechanics parts to fit over the smartphone and the functional optics elements. This design has a problem that they need to redesign the whole-piece optical adaptor for each specific model of smartphone. But in this present invention, the optical adaptor is separated into a holder frame only for fitting a smartphone and a universal optical box containing all the functional parts. For smartphones with different dimensions, as long as the relative positions of the camera and the light source are the same, only the holder frame need to be redesigned, which saves a lot of cost of design and manufacture.

The optical box of the optical adaptor comprises: a receptacle slot which receives and positions the sample in a sample slide in the field of view and focal range of the smartphone camera; a bright-field illumination optics for capturing bright-field microscopy images of a sample; a fluorescent illumination optics for capturing fluorescent microscopy images of a sample; a lever to switch between bright-field illumination optics and fluorescent illumination optics, for example, by sliding the lever inward and outward in the optical box.

In some embodiments, the receptacle slot can have a rubber door attached to it, which can fully cover the slot to prevent the ambient light getting into the optical box to be collected by the camera. In U.S. Pat. Application 2016/0290916, the sample slot is always exposed to the ambient light which won't cause too much problem because it only does bright-field microscopy. But the present invention can take the advantage of this rubber door when doing fluorescent microscopy because the ambient light would bring a lot of noise to the image sensor of the camera.

To capture good fluorescent microscopy image, it is desirable that nearly no excitation light goes into the camera and only the fluorescence emitted by the sample is collected by the camera. For all common smartphones, however, the optical filter putting in front of the camera cannot block the undesired wavelength range of the light emitted from the light source of a smartphone very well due to the large divergence angle of the beams emitted by the light source and the optical filter not working well for un-collimated beams. Collimation optics can be designed to collimate the beam emitted by the smartphone light source to address this issue, but this approach can increase the size and cost of the adaptor. Instead, in this present invention, fluorescent illumination optics enable the excitation light to illuminate the sample partially from the waveguide inside the sample slide and partially from the backside of the sample slide in a large oblique incidence angle so that excitation light will not be collected by the camera to reduce the noise signal getting into the camera.

The bright-field illumination optics in the adaptor receive and turn the beam emitted by the light source to back-illuminate the sample in a normal incidence angle.

Typically, the optical box also comprises a magnifying lens mounted in the box that is aligned with the camera of the smartphone, which magnifies the images captured by the camera. The images captured by the camera can be further processed by the processor of smartphone and outputs the analysis result on the screen of smartphone.

To achieve both bright-field illumination and fluorescent illumination optics in the same optical adaptor, as in the present invention, a slidable lever can be used. The optical elements of the fluorescent illumination optics can be mounted on the lever and when the lever fully slides into the optical box, the fluorescent illumination optics elements block the optical path of bright-field illumination optics and switch the illumination optics to fluorescent illumination optics. When the lever slides out, the fluorescent illumination optics elements mounted on the lever move out of the optical path and switch the illumination optics to bright-field illumination optics. This lever design makes the optical adaptor work in both bright-field and fluorescent illumination modes without the need for designing two different single-mode optical boxes.

The lever comprises two planes at different planes at different heights.

In certain embodiments, two planes can be joined together with a vertical bar and move together in or out of the optical box. In certain embodiments, two planes can be separated and each plane can move individually in or out of the optical box.

The upper lever plane comprises at least one optical element which can be, but not limited to, an optical filter. The upper lever plane moves under the light source and the preferred distance between the upper lever plane and the light source can be, for example, in the range of 0 to 5 mm.

Part of the bottom lever plane is not parallel to the image plane. And the surface of the non-parallel part of the bottom lever plane has mirror finish with high reflectivity larger than 95%. The non-parallel part of the bottom lever plane moves under the light source and deflects the light emitted from the light source to back-illuminate the sample area directly under the camera. The preferred tilt angle of the non-parallel part of the bottom lever plane is in the range of 45 to 65 degrees and the tilt angle is defined as the angle between the non-parallel bottom plane and the vertical plane.

Part of the bottom lever plane can be, for example, parallel to the image plane and can be located under and 1 to 10mm away from the sample. The surface of the parallel part of the bottom lever plane is highly light absorptive with light absorption larger than 95%. This absorptive surface eliminates the reflective light back-illuminating on the sample in small incidence angles.

To slide in and out to switch the illumination optics using the lever, a stopper design comprising a ball plunger and a groove on the lever can be used to stop the lever at a pre-defined position when being pulled outward from the adaptor. This allow the user to use arbitrary force the pull the lever but make the lever to stop at a fixed position where the optical adaptor's working mode is switched to bright-filed illumination.

A sample slider can be mounted inside the receptacle slot to receive the QMAX device (i.e., a preferred sample card) and position the sample in the QMAX device in the field of view and focal range of the smartphone camera.

The sample slider comprises a fixed track frame and a moveable arm:

The track frame can be fixedly mounted in the receptacle slot of the optical box. The track frame can have a sliding track slot that fits the width and thickness of the QMAX device so that the QMAX device can slide along the track. The width and height of the track slot is carefully configured to make the QMAX device shift less than 0.5 mm in the direction perpendicular to the sliding direction in the sliding plane and shift less than 0.2 mm along the thickness direction of the QMAX device.

The track frame has an opened window under the field of view of the camera of smartphone to allow the light to back-illuminate the sample.

A moveable arm can be pre-built in the sliding track slot of the track frame and moves together with the QMAX device to guide the movement of QMAX device in the track frame.

The moveable arm can be equipped with a stopping mechanism with two pre-defined stop positions. For one position, the arm makes the QMAX device stop at the position where a fixed sample area on the QMAX device is directly under the camera of smartphone. For the other position, the arm makes the QMAX device stop at the position where the sample area on QMAX device is out of the field-of-view of the smartphone and the QMAX device can be easily taken out of the track slot.

The moveable arm switches between the two stop positions by a pressing the QMAX device and the moveable arm together to the end of the track slot and then releasing.

The moveable arm can indicate if the QMAX device is inserted in correct direction. The shape of one corner of the QMAX device is configured to be different from the other three right angle corners. The shape of the moveable arm matches the shape of the corner with the special shape so that only when in the correct direction can QMAX device slide to the correct position in the track slot.

C. Smartphone/Detection System

Details of the Smartphone/Detection System are described in a variety of publications including International Application Nos.: PCT/US2016/046437 filed Aug. 10, 2016; and PCT/US2016/051775 filed Sep. 14, 2016; and U.S. Provisional Application Nos.: 62/456065, filed Feb. 7, 2017; 62/456287 and 62/456590, filed Feb. 8, 2017; 62/456504, filed Feb. 8, 2017; U.S. Provisional Application No. 62/459,544, filed Feb. 15, 2017; and 62/460075 and 62/459920, filed Feb. 16, 2017, each are incorporated herein by reference in their entirety.

The disclosed devices, apparatuses, systems, and methods can include or use Q-cards for sample detection, analysis, and quantification. In certain embodiments, the Q-card can be used together with an adaptor that can connect the Q-card with a smartphone detection system. In certain embodiments, the smartphone comprises a camera, an illumination source, or both. In certain embodiments, the smartphone comprises a camera, which can be used to capture images or the sample when the sample is positioned in the field of view of the camera (e.g., by an adaptor). In certain embodiments, the camera includes one set of lenses (e.g., as in iPhone™ 6). In certain embodiments, the camera includes at least two sets of lenses (e.g., as in iPhone™ 7). In certain embodiments, the smartphone comprises a camera, but the camera is not used for image capturing.

In certain embodiments, the smartphone comprises a light source such as but not limited to an LED (light emitting diode). In certain embodiments, the light source can be used to provide illumination to the sample when the sample is positioned in the field of view of the camera (e.g., by an adaptor). In certain embodiments, the light from the light source can be enhanced, magnified, altered, and/or optimized, by optical components of the adaptor.

In certain embodiments, the smartphone comprises a processor that is configured to process the information from the sample. The smartphone can include software instructions that, when executed by the processor, can enhance, magnify, and/or optimize, the signals (e.g., images) from the sample. The processor can include one or more hardware components, such as a central processing unit (CPU), an application-specific integrated circuit (ASIC), an application-specific instruction-set processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction-set computer (RISC), a microprocessor, and the like, or any combination thereof.

In certain embodiments, the smartphone comprises a communication unit, which is configured and/or used to transmit data and/or images related to the sample to another device. Merely by way of example, the communication unit can use a cable network, a wireline network, an optical fiber network, a telecommunications network, an intranet, the Internet, a local area network (LAN), a wide area network (WAN), a wireless local area network (WLAN), a metropolitan area network (MAN), a wide area network (WAN), a public telephone switched network (PSTN), a Bluetooth network, a ZigBee network, a near field communication (NFC) network, and the like, or any combination thereof. In certain embodiments, the smartphone can be, for example, an iPhone™, an Android™ phone, or a Windows™ phone.

D. Method of Manufacture

Details of the Method of Manufacture are described in a variety of publications including International Application No. PCT/US2018/057873 filed Oct. 26, 2018, which is incorporated by reference herein.

Devices of the disclosure can be fabricated using techniques known in the art. The choice of fabrication technique will depend on the material used for the device and the size of the spacer array and/or the size of the spacers. Exemplary materials for fabricating the devices of the invention include glass, silicon, steel, nickel, polymers, e.g., poly(methylmethacrylate) (PMMA), polycarbonate, polystyrene, polyethylene, polyolefins, silicones (e.g., poly(dimethylsiloxane)), polypropylene, cis-polyisoprene (rubber), poly(vinyl chloride) (PVC), poly(vinyl acetate) (PVAc), polychloroprene (neoprene), polytetrafluoroethylene (Teflon), poly(vinylidene chloride) (SaranA), and cyclic olefin polymer (COP) and cyclic olefin copolymer (COC), and combinations thereof. Other materials are known in the art. For example, deep Reactive Ion Etch (DRIE) can be used to fabricate silicon-based devices with small gaps, small spacers and large aspect ratios (ratio of spacer height to lateral dimension). Thermoforming (e.g., embossing, injection molding) of plastic devices can also be used, e.g., when the smallest lateral feature is >20 microns and the aspect ratio of these features is ≤10.

Additional fabrication methods include photolithography (e.g., stereolithography or x-ray photolithography), molding, embossing, silicon micromachining, wet or dry chemical etching, milling, diamond cutting, Lithographie Galvanoformung and Abformung (LIGA), and electroplating. For example, for glass, traditional silicon fabrication techniques of photolithography followed by wet (KOH) or dry etching (reactive ion etching with fluorine or other reactive gas) can be employed. Techniques such as laser micromachining can be adapted for plastic materials with high photon absorption efficiency. This technique is suitable for lower throughput fabrication because of the serial nature of the process. For mass-produced plastic devices, thermoplastic injection molding, and compression molding can be suitable. Conventional thermoplastic injection molding used for mass-fabrication of compact discs (which preserves fidelity of features in sub-microns) can also be employed to fabricate the devices of the invention. For example, the device features are replicated on a glass master by conventional photolithography. The glass master is electroformed to yield a tough, thermal shock resistant, thermally conductive, hard mold. This mold serves as the master template for injection molding or compression molding the features into a plastic device. Depending on the plastic material used to fabricate the devices and the requirements on optical quality and throughput of the finished product, compression molding or injection molding can be chosen as the method of manufacture. Compression molding (also called hot embossing or relief imprinting) has the advantages of being compatible with high molecular weight polymers, which are excellent for small structures and can replicate high aspect ratio structures but has longer cycle times. Injection molding works well for low aspect ratio structures and is most suitable for low molecular weight polymers.

A device can be fabricated in one or more pieces that are then assembled. Layers of a device can be bonded together by clamps, adhesives, heat, anodic bonding, or reactions between surface groups (e.g., wafer bonding). Alternatively, a device with channels or gaps in more than one plane can be fabricated as a single piece, e.g., using stereolithography or other three-dimensional fabrication techniques.

To reduce non-specific adsorption of cells or compounds released by lysed cells onto the surfaces of the device, one or more surfaces of the device can be chemically modified to be non-adherent or repulsive. The surfaces can be coated with a thin film coating (e.g., a monolayer) of commercial non-stick reagents, such as those used to form hydrogels. Additional examples chemical species that can be used to modify the surfaces of the device include, for example, oligoethylene glycols, fluorinated polymers, organosilanes, thiols, poly-ethylene glycol, hyaluronic acid, bovine serum albumin, poly-vinyl alcohol, mucin, poly-HEMA, methacrylated PEG, and agarose. Charged polymers can also be employed to repel oppositely charged species. The type of chemical species used for repulsion and the method of attachment to the surfaces of the device can depend on the nature of the species being repelled and the nature of the surfaces and the species being attached. Such surface modification techniques are known in the art. The surfaces can be functionalized before or after the device is assembled. The surfaces of the device can also be coated to capture certain materials in the sample, e.g., membrane fragments or proteins.

In certain embodiments of the present disclosure, a method for fabricating any Q-Card of the present disclosure can comprise injection molding of the first plate. In certain embodiments of the present disclosure, a method for fabricating any Q-Card of the present disclosure can comprise nanoimprinting or extrusion printing of the second plate. In certain embodiments of the present disclosure, a method for fabricating any Q-Card of the present disclosure can comprise Laser cutting the first plate. In certain embodiments of the present disclosure, a method for fabricating any Q-Card of the present disclosure can comprise nanoimprinting or extrusion printing of the second plate. In certain embodiments of the present disclosure, a method for fabricating any Q-Card of the present disclosure can comprise injection molding and laser cutting the first plate. In certain embodiments of the present disclosure, a method for fabricating any Q-Card of the present disclosure can comprise nanoimprinting or extrusion printing of the second plate. In certain embodiments of the present disclosure, a method for fabricating any Q-Card of the present disclosure can comprise nanoimprinting or extrusion printing to fabricate both the first and the second plate. In certain embodiments of the present disclosure, a method for fabricating any Q-Card of the present disclosure can comprise fabricating the first plate or the second plate, using injection molding, laser cutting the first plate, nanoimprinting, extrusion printing, or a combination thereof. In certain embodiments of the present disclosure, a method for fabricating any Q-Card of the present disclosure can comprise a step of attaching a hinge on the first and the second plates after the fabrication of the first and second plates.

Storage site deposition relates to constructing an area on a plate, wherein the site contains reagents to be added into a sample, and the reagents are capable of being dissolved into the sample that is in contact with the reagents. Various methods known to a skilled artisan can be used to generate or deposit a storage site on a plate, such as, but not limited to, ink-jet deposition, capillary deposition, and photolithographic deposition.

I. Ink-Jet Deposition

Ink-jet printing can be employed for printing biological fluids to form the storage sites. This approach provides very low droplet volumes (e.g., about 100 pL with 1 μm diameter spot size or greater) which minimizes reagents used and cost. Moreover, the printing process can be accelerated to thousands of droplets per second, thereby enabling a high throughput production capability for the QMAX cards. Individual ink-jet devices can be integrated in a modular fashion to enable the printing of multiple fluids. L banks of modular ink-jet devices containing M depositors per module can be assembled in a staggered fashion to print L×M different storage sites on the surface of the plates.

II. Capillary Deposition

Another approach to storage site deposition involves the use of capillary tubing to dispense small amounts of reagent (e.g., capture agent) onto the plate of the QMAX card.

The capillary tubing can be made of fused silica coated with an outer layer of polyimide. These tubes are available commercially in any length with various widths and internal diameters. The preferred dimensions are 80 to 500 μm outer diameter (OD) and 10 to 200 μm inner diameter (ID). The capillary bundles can be affixed to a robot arm and held in a precise pattern by threading the capillaries through array templates. An array template is a structure designed to maintain the capillaries in the desired configuration and spacing, and can consist of, without limitation, a metal grid or mesh, a rigidly-held fabric mesh, a bundle of “sleeve” tubes having an inner diameter sufficient to admit the fluid delivery capillaries, or a solid block having holes or channels, e.g., a perforated aluminum block.

The printing system can print high density reagent arrays covering the bottom surface of microplate wells. To accomplish this, the printing system should maintain a precise printing pattern and accommodate irregular surfaces. Rigid tubes can be used to maintain a precise pattern, however, they cannot readily accommodate irregular surfaces. Flexible tubes will print on uneven surfaces but will not maintain a precise printing pattern. The rigid sleeves, which extend below the aluminum holder assembly approximately 2 cm, support the flexible 190 μm OD fused silica capillary tubing and provide the structural rigidity necessary to maintain a precise grid pattern over this distance. The sleeves also allow the 190 μm tubing to travel smoothly in the Z axis during printing. This ability coupled with the flexibility of the small OD capillary tubing allows for successful printing on surfaces that are not completely flat or absolutely perpendicular to the printing fixture. Since the robot arm extends 0.1 mm to 0.3 mm beyond the point where the capillary bundle contacts the surface, the capillaries flex in the deflection zone resulting in total surface contact among all capillaries in the bundle. When the printing fixture withdraws from the substrate, the capillaries straighten, returning to their original positions. The highly parallel nature of the capillary bundle printing technique allows for microarrays containing from two to over 10,000 chemically unique storage sites to be created with a single “stamp.” The printer can print these arrays at a rate of approximately one per second. This represents a greater than 10-fold increase in speed over existing technologies such as photolithographic in situ synthesis or robotic deposition using conventional load and dispense technology.

Current robotic microarray printing or gridding systems are universally based on various load and dispense techniques. These techniques can be split into two categories. Active loading systems such as syringe needles or capillaries draw up enough solution to dispense multiple storage sites or array elements before returning to reload or collect a new reagent solution. Pin style printing or gridding systems can only print one storage site per pin at a time. The pins are dipped into the reagent solutions momentarily and the amount of solution adhering to the pin is sufficient to print a single storage site. Both categories have limitations that are resolved by the capillary bundle printing system described herein.

In an alternative embodiment, the capillary tubes can be essentially rigid tubes (e.g., stainless steel) mounted in flexible or movable fashion at the attachment site, and slidably held by an array template. In this embodiment, the plurality of capillary tubes can be pressed against a reaction substrate and “even up” at their distal ends by moving lengthwise through the array template, thus accommodating uneven deposition surfaces.

III. Photolithographic Deposition

To increase the spatial resolution and precision of the capillary deposition approach, a combined photolithographic chemical masking and capillary approach is taught herein. The first photolithographic step selectively activates the precise storage site areas on the reaction substrate. Once selective activation has been achieved, the resulting capillary deposition results in uniform storage site distribution.

Many different substrates can be used for this invention, e.g., glass or plastic substrates. With glass substrates, the procedure begins by coating the surface with an aminosilane to aminate the surface. This amine is then reacted with a UV sensitive protecting group, such as the succimidyl ester of α(4,5-dimethoxy-2-nitrobenzyl) referred to as “caged” succimidate. Discrete spots of free amine are revealed on the caged succimidate surface by local irradiation with a UV excitation source (e.g., UV laser or mercury arc). Such a process provides for local storage site modification, surrounded by substrate areas with a relatively high surface tension, unreacted sites.

When using plastic substrates, the procedure begins by coating aminated plastic with an amine blocking group such as a trityl, which is poorly water soluble, and produces a coating with high surface tension. Next, an excitation source (e.g., eximer or IR laser) is used to selectively remove trityl by light-induced heating. The storage site areas are then activated with bifunctional NHS ester or an equivalent. The net result is similar for glass wherein the locally activated storage site areas will have low aqueous surface tension which are surrounded by relatively high surface tension, thereby constraining the capillary dispensing to the storage site area.

E. Sample Types & Subjects

Details of the Samples & Subjects are described in a variety of publications including International Application Nos.: PCT/US2016/046437 filed Aug. 10, 2016; PCT/US2016/051775 ,filed Sep. 14, 2016; PCT/US201/017307 filed Feb. 7, 2018; and PCT/US2017/065440 filed Dec. 8, 2017, each of which is incorporated entirely by reference.

A sample can be obtained from a subject. A subject can be of any age and can be an adult, infant, or child. In some cases, the subject is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between 2 and 20 years old, between 20 and 40 years old, or between 40 and 90 years old). A particular class of subjects that can benefit is subjects who have or are suspected of having an infection (e.g., a bacterial and/or a viral infection). Another particular class of subjects that can benefit is subjects who can be at higher risk of getting an infection. Furthermore, a subject treated by any of the methods or compositions described herein can be male or female. Any of the methods, devices, or kits disclosed herein can also be performed on a non-human subject, such as a laboratory or a farm animal. Non-limiting examples of a non-human subjects include, for example, a dog, a goat, a guinea pig, a hamster, a mouse, a pig, a non-human primate (e.g., a gorilla, an ape, an orangutan, a lemur, or a baboon), a rat, a sheep, a cow, or a zebrafish.

The disclosed devices, apparatus, systems, and methods can be used for samples such as but not limited to diagnostic samples, clinical samples, environmental samples and foodstuff samples.

For example, in certain disclosed embodiments, the devices, apparatus, systems, and methods can be used for a sample that includes cells, tissues, bodily fluids, and/or a mixture thereof. In certain embodiments, the sample comprises a human body fluid. In certain embodiments, the sample comprises at least one of cells, tissues, bodily fluids, stool, amniotic fluid, aqueous humour, vitreous humour, blood, whole blood, fractionated blood, plasma, serum, breast milk, cerebrospinal fluid, cerumen, chyle, chime, endolymph, perilymph, feces, gastric acid, gastric juice, lymph, mucus, nasal drainage, phlegm, pericardial fluid, peritoneal fluid, pleural fluid, pus, rheum, saliva, sebum, semen, sputum, sweat, synovial fluid, tears, vomit, urine, and exhaled breath condensate.

In certain embodiments, the devices, apparatus, systems, and methods disclosed are used for an environmental sample that is obtained from any suitable source, such as but not limited to: river, lake, pond, ocean, glaciers, icebergs, rain, snow, sewage, reservoirs, tap water, drinking water, and like sources; solid samples from soil, compost, sand, rocks, concrete, wood, brick, sewage, and like samples; and gaseous samples from the air, underwater heat vents, industrial exhaust, vehicular exhaust, and like samples. In certain embodiments, the environmental sample is fresh from the source. In certain embodiments, the environmental sample is processed. For example, samples that are not in liquid form can be converted to liquid form before the subject devices, apparatus, systems, and methods are applied.

In certain embodiments, the devices, apparatus, systems, and methods disclosed are used for a foodstuff sample, which is suitable or has the potential to become suitable for animal consumption, e.g., human consumption. In certain embodiments, a foodstuff sample includes raw ingredients, cooked or processed food, plant and animal sources of food, preprocessed food as well as partially or fully processed food, and like samples. In certain embodiments, samples that are not in liquid form can be converted to liquid form before the subject devices, apparatus, systems, and methods are applied.

The subject devices, apparatus, systems, and methods can be used to analyze any volume of the sample. Examples of the volumes include, but are not limited to, about 10 mL or less, 5 mL or less, 3 mL or less, 1 microliter (“uL”) or less, 500 uL or less, 300 uL or less, 250 uL or less, 200 uL or less, 170 uL or less, 150 uL or less, 125 uL or less, 100 uL or less, 75 uL or less, 50 uL or less, 25 uL or less, 20 uL or less, 15 uL or less, 10 uL or less, 5 uL or less, 3 uL or less, 1 uL or less, 0.5 uL or less, 0.1 uL or less, 0.05 uL or less, 0.001 uL or less, 0.0005 uL or less, 0.0001 uL or less, 10 pL or less, 1 pL or less, including intermediate values and ranges.

In certain embodiments, the volume of the sample includes, but is not limited to, about 100 uL or less, 75 uL or less, 50 uL or less, 25 uL or less, 20 uL or less, 15 uL or less, 10 uL or less, 5 uL or less, 3 uL or less, 1 uL or less, 0.5 uL or less, 0.1 uL or less, 0.05 uL or less, 0.001 uL or less, 0.0005 uL or less, 0.0001 uL or less, 10 pL or less, 1 pL or less, or a range between any two of the values. In certain embodiments, the volume of the sample includes, but is not limited to, about 10 uL or less, 5 uL or less, 3 uL or less, 1 uL or less, 0.5 uL or less, 0.1 uL or less, 0.05 uL or less, 0.001 uL or less, 0.0005 uL or less, 0.0001 uL or less, 10 pL or less, 1 pL or less, including intermediate values and ranges.

In certain embodiments, the amount of the sample can be, for example, about a drop of liquid. In certain embodiments, the amount of sample can be, for example, the amount collected from a pricked finger or fingerstick. In certain embodiments, the amount of sample can be, for example, the amount collected from a microneedle, micropipette, or a venous draw.

F. Machine Learning

Details of the Network are described in a variety of publications including International Application Nos.: PCT/US2018/017504 filed Feb. 8, 2018, and PCT/US2018/057877 filed Oct. 26, 2018, each of which are incorporated by reference.

One aspect of the present invention provides a framework of machine learning and deep learning for analyte detection and localization. A machine learning algorithm is an algorithm that is able to learn from data. A more rigorous definition of machine learning is “A computer program is said to learn from experience E with respect to some class of tasks T and performance measure P, if its performance at tasks in T, as measured by P, improves with experience E.” It explores the study and construction of algorithms that can learn from and make predictions on data—such algorithms overcome the static program instructions by making data driven predictions or decisions, through building a model from sample inputs.

Deep learning is a specific kind of machine learning based on a set of algorithms that attempt to model high level abstractions in data. In a simple case, there might be two sets of neurons: ones that receive an input signal and ones that send an output signal. When the input layer receives an input, it passes on a modified version of the input to the next layer. In a deep network, there are many layers between the input and output (and the layers are not made of neurons but it can help to think of it that way), allowing the algorithm to use multiple processing layers, composed of multiple linear and non-linear transformations.

One aspect of the present invention is to provide two analyte detection and localization approaches. The first approach is a deep learning approach and the second approach is a combination of deep learning and computer vision approaches.

I. Deep Learning Approach

In the first approach, the disclosed analyte detection and localization workflow consists of two stages, training and prediction. We describe training and prediction stages in the following paragraphs.

(i) Training Stage

In the training stage, training data with annotation is fed into a convolutional neural network. A convolutional neural network is a specialized neural network for processing data that has a grid-like, feed forward and layered network topology. Examples of the data include time-series data, which can be thought of as a 1D grid taking samples at regular time intervals, and image data, which can be thought of as a 2D grid of pixels. Convolutional networks have been successful in practical applications. The name “convolutional neural network” indicates that the network employs a mathematical operation called convolution. Convolution is a specialized kind of linear operation. Convolutional networks are simply neural networks that use convolution in place of general matrix multiplication in at least one of their layers.

The machine learning model receives one or multiple images of samples that contain the analytes taken by the imager over the sample holding QMAX device as training data. Training data are annotated for analytes to be assayed, wherein the annotations indicate whether or not analytes are in the training data and where they locate in the image. Annotation can be done in the form of tight bounding boxes which fully contains the analyte, or center locations of analytes. In the latter case, center locations are further converted into circles covering analytes or a Gaussian kernel in a point map.

When the size of training data is large, training a machine learning model presents two challenges: annotation (usually done by human) is time consuming, and the training is computationally expensive. To overcome these challenges, one can partition the training data into patches of small size, then annotate and train on these patches, or a portion of these patches. The term “machine learning” can refer to algorithms, systems, and apparatus in the field of artificial intelligence that often use statistical techniques and a artificial neural network trained from data without being explicitly programmed.

The annotated images can be fed to the machine learning (ML) training module, and the model trainer in the machine learning module can train a ML model from the training data (annotated sample images). The input data can be fed to the model trainer in multiple iterations until certain stopping criterion is satisfied. The output of the ML training module is a ML model - a computational model that is built from a training process in the machine learning from the data that gives computer the capability to perform certain tasks (e.g., detect and classify the objects) on its own.

The trained machine learning model is applied during the predication (or inference) stage by the computer. Examples of machine learning models include ResNet, DenseNet, and like models, which are also named as “deep learning models” because of the depth of the connected layers in their network structure. In certain embodiments, the Caffe library with fully convolutional network (FCN) was used for model training and predication, and other convolutional neural network architectures and a library can also be used, such as TensorFlow.

The training stage generates a model that will be used in the prediction stage. The model can be repeatedly used in the prediction stage for assaying the input. Thus, the computing unit only needs access to the generated model. It does not need access to the training data, nor requiring the training stage to be run again on the computing unit.

(ii) Prediction Stage

In the predication/inference stage, a detection component is applied to the input image, and an input image is fed into the predication (inference) module preloaded with a trained model generated from the training stage. The output of the prediction stage can be bounding boxes that contain the detected analytes with their center locations or a point map indicating the location of each analyte, or a heatmap that contains the information of the detected analytes.

When the output of the prediction stage is a list of bounding boxes, the number of analytes in the image of the sample for assaying is characterized by the number of detected bounding boxes. When the output of the prediction stage is a point map, the number of analytes in the image of the sample for assaying is characterized by the integration of the point map. When the output of the prediction is a heatmap, a localization component is used to identify the location and the number of detected analytes is characterized by the entries of the heatmap.

One embodiment of the localization algorithm is to sort the heatmap values into a one-dimensional ordered list, from the highest value to the lowest value. Then pick the pixel with the highest value, remove the pixel from the list, along with its neighbors. Iterate the process to pick the pixel with the highest value in the list, until all pixels are removed from the list.

In the detection component using heatmap, an input image, along with the model generated from the training stage, is fed into a convolutional neural network, and the output of the detection stage is a pixel-level prediction, in the form of a heatmap. The heatmap can have the same size as the input image, or it can be a scaled down version of the input image, and it is the input to the localization component. We disclose an algorithm to localize the analyte center. The main idea is to iteratively detect local peaks from the heatmap. After the peak is localized, we calculate the local area surrounding the peak but with smaller value. We remove this region from the heatmap and find the next peak from the remaining pixels. The process is repeated until only all pixels are removed from the heatmap.

In certain embodiments, the present invention provides the localization algorithm to sort the heatmap values into a one-dimensional ordered list, from the highest value to the lowest value. Then pick the pixel with the highest value, remove the pixel from the list, along with its neighbors. Iterate the process to pick the pixel with the highest value in the list, until all pixels are removed from the list.

```
Algorithm GlobalSearch (heatmap)
Input:
    heatmap
Output:
    loci
loci ←{ }
sort(heatmap)
while (heatmap is not empty) {
  s ← pop(heatmap)
  D ← {disk center as s with radius R}
  heatmap = heatmap \ D // remove D from the heatmap
  add s to loci
}
```

After sorting, heatmap is a one-dimensional ordered list, where the heatmap value is ordered from the highest to the lowest. Each heatmap value is associated with its corresponding pixel coordinates. The first item in the heatmap is the one with the highest value, which is the output of the pop(heatmap) function. One disk is created, where the center is the pixel coordinate of the one with highest heatmap value. Then all heatmap values whose pixel coordinates resides inside the disk is removed from the heatmap. The algorithm repeatedly pops up the highest value in the current heatmap, removes the disk around it, until the items are removed from the heatmap.

In the ordered list heatmap, each item has the knowledge of the proceeding item, and the following item. When removing an item from the ordered list, we make the following changes: Assume the removing item is xᵣ, its preceding item is xₚ, and its following item is x_{f}.
For the preceding item xₚ, re-define its following item to the following item of the removing item. Thus, the following item of xₚ is now x_{f}.
For the removing item xᵣ, un-define its proceeding item and following item, which removes it from the ordered list.
For the following item x_{f}, re-define its proceeding item to the proceeding item of the removed item. Thus, the proceeding item of x_{f} is now xₚ.

After all items are removed from the ordered list, the localization algorithm is complete. The number of elements in the set loci will be the count of analytes, and location information is the pixel coordinate for each s in the set loci.

Another embodiment searches local peak, which is not necessarily the one with the highest heatmap value. To detect each local peak, we start from a random starting point, and search for the local maximal value. After we find the peak, we calculate the local area surrounding the peak but with smaller value. We remove this region from the heatmap and find the next peak from the remaining pixels. The process is repeated until only all pixels are removed from the heatmap.

```
Algorithm LocalSearch (s, heatmap)
Input:
  s: starting location (x, y)
  heatmap
Output:
  s: location of local peak.
We only consider pixels of value > 0.
Algorithm Cover (s, heatmap)
Input:
  s: location of local peak.
  heatmap:
Output:
  cover: a set of pixels covered by peak:
```

This is a breadth-first-search algorithm starting from s, with one altered condition of visiting points: a neighbor p of the current location q is only added to cover if heatmap[p]>0 and heatmap[p]<=heatmap[q]. Therefore, each pixel in cover has a non-descending path leading to the local peak s.

```
Algorithm Localization (heatmap)
Input:
    heatmap
Output:
    loci
loci ←{ }
pixels ←{all pixels from heatmap}
while pixels is not empty {
  s ←any pixel from pixels
  s ←LocalSearch(s, heatmap)  // s is now local peak
  probe local region of radius R surrounding s for better local peak
  r ←Cover(s, heatmap)
  pixels ← pixels \ r  // remove all pixels in cover
  add s to loci
```

II. Mixture of Deep Learning and Computer Vision Approaches

In the second approach, the detection and localization are realized by computer vision algorithms, and a classification is realized by deep learning algorithms, wherein the computer vision algorithms detect and locate possible candidates of analytes, and the deep learning algorithm classifies each possible candidate as a true analyte and false analyte. The location of all true analyte (along with the total count of true analytes) are recorded as the output.

(i) Detection. The computer vision algorithm detects possible candidates based on the characteristics of analytes, including, for example, intensity, color, size, shape, distribution, and like considerations. A pre-processing scheme can improve the detection. Pre-processing schemes can include, for example, contrast enhancement, histogram adjustment, color enhancement, de-nosing, smoothing, de-focus, and like schemes. After pre-processing, the input image can be sent to a detector. The detector indicates the existence of possible candidates for analytes and gives an estimate of analyte location. The detection can be based on the analyte structure (e.g., edge, line, circle, and like detections), the connectivity (e.g., blob, connect components, contour, and like detections), intensity, color, shape using schemes such as adaptive thresholding, and like schemes.

(ii) Localization. After detection, the computer vision algorithm locates each possible candidate of analytes by providing its boundary or a tight bounding box containing it. This can be achieved through object segmentation algorithms, such as adaptive thresholding, background subtraction, flood-fill, mean shift, watershed, and like algorithms. Very often, the localization can be combined with detection to produce the detection results along with the location of each possible candidates of analytes.

(iii) Classification. The deep learning algorithms, such as convolutional neural networks, achieve start-of-the-art visual classification. We employ deep learning algorithms for classification on each possible candidate of analytes. Various convolutional neural network can be utilized for analyte classification, such as VGGNet, ResNet, MobileNet, DenseNet, and like networks.

Given each possible candidate of analyte, the deep learning algorithm computes through layers of neurons via convolution filters and non-linear filters to extract high-level features that differentiate an analyte against a non-analyte. A layer of a fully convolutional network will combine high-level features into classification results, which tells whether it is a true analyte or not, or the probability of being a analyte.

Other Embodiments

Further examples of inventive subject matter according to the present disclosure are described in the following enumerated paragraphs.

It must be noted that, as used herein and in the appended claims, the singular forms “a”, “an”, and “the” include plural referents unless the context clearly dictates otherwise, e.g., when the word “single” is used. For example, reference to “an analyte” includes a single analyte and multiple analytes, reference to “a capture agent” includes a single capture agent and multiple capture agents, reference to “a detection agent” includes a single detection agent and multiple detection agents, and reference to “an agent” includes a single agent and multiple agents.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated “about” means within an acceptable error range for the particular value should be assumed. The term “about” has the meaning as commonly understood by one of ordinary skill in the art. In some embodiments, “about” refers to ±10%. In some embodiments, “about” refers to ±5%.

As used herein, the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function. Similarly, subject matter that is recited as being configured to perform a particular function can additionally or alternatively be described as being operative to perform that function.

As used herein, “for example,” “as an example,” and/or “example” and “exemplary” when used with reference to one or more components, features, details, structures, embodiments, and/or methods, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

As used herein, “at least one of” and “one or more of,” in reference to a list of more than one entity, means any one or more of the entity in the list of entity, and is not limited to at least one of each and every entity specifically listed within the list of entity. For example, “at least one of A and B” (or, equivalently, “at least one of A or B,” or, equivalently, “at least one of A and/or B”) can refer to A alone, B alone, or the combination of A and B.

As used herein, “and/or” placed between a first entity and a second entity means one of (1) the first entity, (2) the second entity, and (3) the first entity and the second entity. Multiple entity listed with “and/or” should be construed in the same manner, i.e., “one or more” of the entity so conjoined. Other entities can optionally be present other than the entity specifically identified by the “and/or” clause, whether related or unrelated to those entities specifically identified.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art.

In the event that any patents, patent applications, or other references are incorporated by reference and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

1. A method for performing two assays in different portions of a same sample without using a physical barrier, comprising:
(a) having two plates facing each other and separated by a gap G, wherein each of the plate has a sample contact area for contacting a sample that contains or is suspected containing an analyte;
(b) having, the sample contact area, reagent A in an area A and reagent B in an area A, respectively, wherein the area A and area B is separated by a distance D;
(c) confining the sample between the sample contact areas of the two plates forming a thin layer, wherein the sample has a thickness confined by the gap G that is smaller than the distance D and wherein there is no physical barrier between the area A and B ; and
(d) observing the reaction of reagents in area A and area B within a time period that is shorter than the diffusion time of the analyte, the reagent in area A and the reagent in area B diffuse across the distance D.

2. The method and the device of any prior claim 1, wherein the two plates are movable relative to each other, wherein the gap between the two plates are regulated by spacers, wherein at least one of the spacers is in the sample contact area.

3. A device for analyzing a sample, comprising:
a. a first plate, comprising:
i. a first storage site having a first reagent configured to bind to a first analyte; and
ii. a second storage site having a second reagent configured to bind to a second analyte; and
b. a second plate movable relative to said first plate into different configurations including an open configuration and a closed configuration,
wherein, in the open configuration, the first plate and the second plate are at least partially separated, and at least one of the first plate and the second plate receives deposition of a sample containing or suspected of containing the first analyte and the second analyte,
wherein, in the closed configuration, at least a portion of the deposited sample is compressed into a layer of uniform thickness in contact with the first plate and the second plate to form a sample thickness,
wherein the first storage site and the second storage site are fluidically connected by the deposited sample and are separated from each other by a separation distance, and the separation distance is greater than:
a distance that the first reagent contained in the first storage site can diffuse to the second storage site within a period of time, or
(ii) a distance that the second reagent contained in the second storage site can diffuse to the first storage site within a period of time.

4. A device for analyzing a sample, comprising:
a. a first plate, comprising:
i. a first storage site having a first reagent configured to bind to a first analyte; and
ii. a second storage site having a second reagent configured to bind to a second analyte; and
b. a second plate movable relative to said first plate into different configurations including an open configuration and a closed configuration,
wherein, in the open configuration, the first plate and the second plate are at least partially separated, and at least one of the first plate and the second plate receives deposition of a sample containing or suspected of containing the first analyte and the second analyte,
wherein, in the closed configuration, at least a portion of the deposited sample is compressed into a layer of uniform thickness in contact with the first plate and the second plate to form a sample thickness,
wherein the first storage site and the second storage site are fluidically connected by the deposited sample and are separated from each other by a separation distance, and wherein the separation distance is governed by the formula of:
Separation Distance≥Sample Thickness.

5. A device for analyzing a sample, comprising:
a. a first plate, comprising a first storage site having a first reagent configured to bind to a first analyte; and
b. a second plate, comprising a second storage site having a second reagent configured to bind to a second analyte, wherein said second plate is movable relative to said first plate into different configurations including an open configuration and a closed configuration,
wherein, in the open configuration, the first plate and the second plate are at least partially separated, and at least one of the first plate and the second plate receives deposition of a sample containing or suspected of containing the first analyte and the second analyte,
wherein, in the closed configuration, at least a portion of the deposited sample is compressed into a layer of uniform thickness in contact with the first plate and the second plate to form a sample thickness,
wherein the first storage site and the second storage site are fluidically connected by the deposited sample and are separated from each other by a separation distance, and the separation distance is greater than:
a. a distance that the first reagent contained in the first storage site can diffuse to the second storage site within a period of time, or
b. a distance that the second reagent contained in the second storage site can diffuse to the first storage site within a period of time.

6. A device for analyzing a sample, comprising:
a. a first plate, comprising a first storage site having a first reagent configured to bind to a first analyte; and
b. a second plate, comprising a second storage site having a second reagent configured to bind to a second analyte, wherein said second plate is movable relative to said first plate into different configurations including an open configuration and a closed configuration,
wherein, in the open configuration, the first plate and the second plate are at least partially separated, and at least one of the first plate and the second plate receives deposition of a sample containing or suspected of containing the first analyte and the second analyte,
wherein, in the closed configuration, at least a portion of the deposited sample is compressed into a layer of uniform thickness in contact with the first plate and the second plate to form a sample thickness,
wherein the first storage site and the second storage site are fluidically connected by the deposited sample and are separated from each other by a separation distance, and wherein the separation distance is governed by the formula:
Separation Distance≥Sample Thickness.

7. The device of any one of claims 3-6, wherein at least one of the plates is transparent.

8. The device of any one of claims 3-6, wherein the first plate and the second plate are made of a material selected from polystyrene, PMMA, PC, COC, COP, or a combination thereof

9. The device of any one of claims 3-6, wherein at least one of the first plate and the second plate have a thickness in the range of 20 μm to 250 μm.

10. The device of any one of claims 3-6, wherein at least one of the first plate and the second plate have a Young's modulus in the range 0.1 to 5 GPa.

11. The device of any one of claims 3-6, wherein the thickness of the first plate or the second plate times the Young's modulus of the first plate or the second plate is in the range 60 to 750 GPa-um.

12. The device of any one of claims 3-6, wherein the layer of uniform thickness is uniform over a lateral area that is at least 1 mm².

13. The device of any one of claims 3-6, wherein the layer of uniform thickness has a sample thickness uniformity of up to +/−5%.

14. The device of any one of claims 3-6, wherein the uniform thickness between the first plate and the second plate, in the closed configuration, is less than 200 μm.

15. The device of any one of claims 3-6, wherein the uniform thickness between the first plate and the second plate, in the closed configuration, is from 1 μm to 30 μm.

16. The device of any one of claims 3-6, wherein at least one of the plates has a plurality of spacers affixed to the surface of the plate.

17. The device of claim 16, wherein the spacers are made of a material selected from polystyrene, PMMA, PC, COC, COP, or a combination thereof

18. The device of claim 16, wherein the spacers have a pillar shape.

19. The device of claim 16, wherein the spacers have a pillar shape, and the sidewall corners of the spacers have a round shape with a radius of curvature at least 1 μm.

20. The device of claim 16, wherein the spacers are pillars with a cross-sectional shape selected from round, polygonal, circular, triangular, square, rectangular, oval, elliptical, or a super-positional combination thereof.

21. The device of claim 16, wherein the spacers have a substantially flat top surface.

22. The device of claim 16, wherein the spacers have a substantially uniform cross-section.

23. The device of claim 16, wherein the spacers have a predetermined substantially uniform height.

24. The device of claim 16, wherein the spacers have a constant inter-spacer distance.

25. The device of claim 24, wherein the constant inter-spacer distance is from about 1 μm to 120 μm.

26. The device of claim 16, wherein the spacers have a periodic inter-spacer distance.

27. The device of claim 16, wherein, for each spacer, the ratio of the lateral dimension of the spacer to its height is at least 1.

28. The device of claim 16, wherein the spacers have a filling factor of 1% or higher, wherein the filling factor is the ratio of the lateral spacer contact area to the total lateral plate area of one plate.

29. The device of claim 16, wherein the Young's modulus of the spacers times filling factor of the spacers is equal or larger than 20 MPa, wherein the filling factor is the ratio of the lateral spacer contact area to the total lateral plate area of one plate

30. The device of claim 16, wherein the spacers have a density of at least 1,000/mm².

31. The device of any one of claims 3-6, wherein the first analyte and the second analyte are the same.

32. The device of any one of claims 3-6, wherein the first reagent comprises a cell lysis agent.

33. The device of claim 4, wherein the cell lysis agent is selected from an ethoxylated nonylphenol non-ionic surfactant, sodium deoxycholate, sodium dodecyl sulfate, ammonium-chloride-potassium (ACK) buffer, a zwitterionic detergent of the formula C₁₉H₄₁NO₃S, a zwitterionic detergent, or a saponin.

34. The device of any one of claims 3-6, wherein the first reagent is configured to perform a first assay, and the second reagent is configured to perform a second assay.

35. The device of any one of claims 3-6, wherein the first assay and the second assay are independently selected from an optical assay, a colorimetric assay, a mercurimetric assay, a luminescent assay, an electro-luminescent assay, a chemical-luminescent assay, a nonlinear optical assay, an electrical assay, a capacitive measurement assay, a resistive measurement assay, an impedance measurement assay, a chemical assay, and a mechanical assay.

36. The device of any one of claims 3-6, wherein the device further comprises a third storage site comprising a third reagent for binding to a third target analyte.

37. The device of claim 36, wherein the third storage site is separated from the first storage site and the second storage site by the separation distance.

38. The device of claim 36, wherein the third storage site is fluidically connected to the first storage site and the second storage site by the deposited sample.

39. The device of any one of claims 3-6, wherein the period of time is 1 second, 5 seconds, 10 seconds, 20 seconds, 30 seconds, 40 seconds, 50 seconds, 1 minute, 2 minutes, 3 minutes, 4 minutes, 5 minutes, 10 minutes, 15 minutes, 30 minutes, or greater than 30 minutes, including intermediate values and ranges.

40. The device of any one of claims 3-6, wherein the separation distance is 5 micrometers (um), 10 um, 25 um, 50 um, 100 um, 150 um, 200 um, 250 um, 300 um, 400 um, 500 um, 750 um, 1 millimeter, 2 mm, 3 mm, 4 mm, or 5 mm, including intermediate values and ranges.

41. The device of any one of claims 3-6, wherein the second storage site surrounds the first storage site.

42. The device of any one of claims 3-6, wherein the first storage site and the second storage site have a shape that are independently selected from round, polygonal, circular, triangular, square, rectangular, pentagonal, hexagonal, oval, elliptical, or a combination thereof.

43. The device of any one of claims 3-6, wherein the separation distance is governed by the formula of: Separation Distance≥2×Sample Thickness.

44. The device of any one of claims 3-6, wherein the separation distance is governed by the formula of: Separation Distance≥5×Sample Thickness.

45. The device of any one of claims 3-6, wherein the separation distance is governed by the formula of: Separation Distance≥10×Sample Thickness.

46. A method for analyzing a liquid sample, comprising:
(a) obtaining a sample that contains or is suspected to contain a first analyte and a second analyte;
(b) obtaining the device of claim 3 or 4;
(c) depositing the sample on one or both of the plates when the plates are configured in an open configuration, wherein the open configuration is a configuration in which the two plates are either partially or completely separated apart and the spacing between the plates is not regulated by the spacers;
(d) after (c), bringing the two plates together into a closed configuration; in which at least part of the sample forms a layer of substantially uniform thickness that is confined by the sample contact surfaces of the plates, wherein the substantially uniform thickness of the layer is regulated by the spacers and the plates; and
(e) analyzing at least one of the first analyte and the second analyte in the layer of uniform thickness while the plates are in the closed configuration.

47. A method for analyzing a liquid sample, comprising:
(a) obtaining a sample that contains or is suspected to contain a first analyte and a second analyte;
(b) obtaining a device comprising:
i. a first plate;
ii. a second plate; and
iii. a first storage site comprising a first reagent and a second storage site comprising a second reagent, wherein each of the first storage site and the second storage site are (A) disposed on one of the first plate and the second plate, and (B) fluidically connected by a sample;
(c) depositing the sample on one or both of the plates when the plates are configured in an open configuration, wherein the open configuration is a configuration in which the two plates are either partially or completely separated apart and the spacing between the plates is not regulated by the spacers;
(d) after (c), bringing the two plates together into a closed configuration; in which at least part of the sample forms a layer of substantially uniform thickness that is confined by the sample contact surfaces of the plates, wherein the substantially uniform thickness of the layer is regulated by the spacers and the plates; and
(e) analyzing at least one of the first analyte and the second analyte in the layer of uniform thickness while the plates are in the closed configuration,
wherein the analyzing comprises (i) analyzing one or more first images from a first field of view within the first storage site, and (ii) analyzing one or more second images from a second field of view within the second storage site, wherein the first field of view and the second field of view are separated by a separation distance, and wherein the separation distance is greater than:
(a) a distance that the first reagent can diffuse to the second field of view within a period of time, or
(b) a distance that the second reagent can diffuse to the first field of view within a period of time.

48. The method of claim 46 or 47, further comprising determining a relevant volume of sample, wherein the relevant volume is a product of a predetermined area and a thickness of the layer of uniform thickness at the closed configuration.

49. The method of claim 46, further comprising determining a concentration of first analyte and/or the second analyte in a relevant volume of sample.
